(19) United States
(12) Patent Application Publication
(10) Pub. No.: US 2020/0179369 A1
(43) Pub. Date: Jun. 11, 2020

(54) TREATMENT OF DIABETIC NEPHROPATHY

(71) Applicant: TAKEDA GMBH, Konstanz (DE)

(72) Inventors: Guido HANAUER, Konstanz (DE); Stephanie VOLLERT, Bad Segeberg (DE); Masatoshi HAZAMA, Fujisawa-shi, Kanagawa (JP); Takanori MATSUO, Fujisawa-shi, Kanagawa (JP)

(21) Appl. No.: 15/746,560

(22) PCT Filed: Jul. 28, 2016
(86) PCT No.: PCT/EP2016/067968

§ 371 Date: Jan. 22, 2018

Related U.S. Application Data
(60) Provisional application No. 62/198,213, filed on Jul. 29, 2015.

(51) Int. Cl. A61K 31/473; A61K 31/4184; A61K 31/4245; A61K 9/00; A61P 13/12

(52) U.S. Cl. CPC A61K 31/473; A61K 31/4184; A61P 13/12; A61K 9/0053; A61K 31/4245

(57) ABSTRACT

The present invention is directed to the treatment of diabetic nephropathy with a) a phosphodiesterase 4 inhibitor, b) a combination of a phosphodiesterase 4 inhibitor with an AT₁ angiotensin II receptor antagonist or c) a combination of a phosphodiesterase 4 inhibitor with an angiotensin-converting enzyme inhibitor.

### FIELD OF THE INVENTION

The present invention is directed to the treatment of diabetic nephropathy. More particularly, the present invention is directed to the treatment of diabetic nephropathy with a) a phosphodiesterase 4 inhibitor (sometimes abbreviated as PDE4 inhibitor in the present specification), b) a combination of a phosphodiesterase 4 inhibitor with an AT₁ angiotensin II receptor antagonist or c) a combination of a phosphodiesterase 4 inhibitor with an angiotensin-converting enzyme inhibitor.

### BACKGROUND OF THE INVENTION

The International Diabetes Federation estimates that the worldwide prevalence of diabetes will increase from 285 million persons in 2010 to 439 millions in 2030, a relative increase of 50% (Shaw J E, Diabetes Res Clin Pract 2010, Vol 87, pp 4-14). Among patients with diabetes mellitus type 1, the incidence of diabetic nephropathy has apparently decreased from 30-35% in the cohorts who developed diabetes 40 to 50 years ago to 10-15% in recent cohorts (Bojestig M, N Engl J Med 1994, Vol 330, pp 15-18; Hovind P Diabetes Care 2003, Vol 26, pp 1258-1264). However, due to the increase in diabetes mellitus type 2, the absolute prevalence of diabetic nephropathy has increased over the past two decades. In the United States in 2009, diabetic nephropathy was reported to be the cause of 44% of all cases of end-stage renal disease (ESRD), with an incidence of 155 diabetic patients developing ESRD per million per year. A modest decrease has been noted nowadays in the number of patients with diabetes mellitus type 2 who develop ESRD both in the United States and Europe (Burrows N R Diabetes Care 2010, Vol 33, pp 73-77; Zocalli C Clin J Am Soc Nephrol 2009, Vol 4, pp S18-S22).

Diabetic nephropathy is characterized by an injury of the kidney accompanied by a progressive increase in the levels of albuminuria, hypertension, glomerulosclerosis and the eventual reduction in glomerular filtration rate (GFR), leading to ESRD.

Early diabetic nephropathy is defined as persistent microalbuminuria measured on at least two different occasions as an albumin excretion rate of 20 to 200 μg/min or 30 to 300 mg/24 h (for example: “Programm für nationale VersorgungsLeitlinien Germany 2010). Typically today, clinical centers most probably would use spot urine measurements of the albumin-to-creatinine ratio to define the existence of microalbuminuria. The criteria for the existence of microalbuminuria based on the albumin-to-creatinine ratio vary somewhat from country to country, but are usually in the range of 2.5 to 25 mg/mmol (or 20-200 mg/g) for men and 3.5-35 mg/mmol (or 30-300 mg/g) for women. Overt diabetic nephropathy is defined as albumin excretion beyond the microalbuminuric range, or urinary protein excretion >500mg/24 h.

According to today's knowledge about the disease, diabetic nephropathy in diabetes mellitus type 1 patients usually takes more than 5 to 10 years to develop, whereas in diabetes mellitus type 2 patients it may be present already at the time of diabetes mellitus type 2 diagnosis, due to several years of undiagnosed hyperglycemia.

The main risk factors for developing as well as worsening diabetic nephropathy are hyperglycemia, hypertension, albumin excretion rate, smoking, increased BMI, lipid abnormalities (increased levels of LDL-cholesterin and triglycerides, decreased levels of HDL-cholesterin), advanced age, male, overall length of diabetes disease, genetic predisposition, positive family anamneses of hypertension or nephropathy and ethnicity, of which hyperglycemia, hypertension, albumin excretion rate, smoking, increased BMI and lipid abnormalities can be therapeutically influenced.

Apart from lifestyle modifications, as for example, achieving or maintaining a healthy weight (BMI 20 to 25), lowering salt intake, undertaking a frequent exercise program and limiting the alcohol intake, the treatment recommendations for diabetic patients at risk of developing diabetic nephropathy or already being diagnosed with diabetic nephropathy include
treating these patients whose blood pressure is consistently >140 mm Hg systolic or >90 mm Hg diastolic with blood pressure lowering agents, such as an ARB (angiotensin II receptor blocker or antagonist) or ACE-I (angiotensin I converting enzyme inhibitor) to maintain a blood pressure that is consistently ≤140 mm Hg systolic and ≤90mm Hg diastolic independently therefrom whether the urine albumin excretion in these patients is <30 mg/24 h, between 30 to 300 mg/24 h or >300mg/24 h
treating these patients with HbA1c lowering agents to target a hemoglobin A1c of 7.0% in those patients that are not at risk of hypoglycemia
treating patients with HbA1c lowering agents to target a hemoglobin A1c of slightly above 7.0% in those patients with co-morbidities or limited life expectancy and risk of hypoglycemia.

Although modern treatment of diabetes mellitus and increased blood pressure to some degree have stabilized the occurrence of diabetic nephropathy, the condition is still a leading cause in patients who require dialysis and transplantation in the Western World. Thus, there is still a high demand for novel and effective alternative medicaments for the treatment (and prevention) of all stages of diabetic nephropathy.

### SUMMARY OF THE INVENTION

In a first aspect the present invention provides a method for the treatment of diabetic nephropathy in a mammal in need of such treatment, including administering to the mammal suffering from diabetic nephropathy a therapeutically effective amount of phosphodiesterase 4 inhibitor.

The phosphodiesterase 4 inhibitor is selected from 5-((2R,4aR,10bR)-9-Ethoxy-2-hydroxy-8-methoxy-1,2,3,4,4a,10b-hexahydro-phenanthridin-6-yl)1-methyl-1H-pyridin-2-one (hereinafter referred to as “Compound A”) and a pharmaceutically acceptable salt thereof.

In a second aspect the present invention provides a method for the treatment of diabetic nephropathy in a mammal in need of such treatment, including administering to the mammal suffering from diabetic nephropathy a therapeutically effective amount of a combination of Compound A or a pharmaceutically acceptable salt thereof and a AT₁ angiotensin II receptor antagonist.

In an embodiment of the second aspect Compound A or a pharmaceutically acceptable salt thereof and the AT₁ angiotensin II receptor antagonist are administered in one single dosage form.

In another embodiment of the second aspect Compound A or a pharmaceutically acceptable salt thereof and the AT₁ angiotensin II receptor antagonist are administered concurrently or sequentially in two separate dosage forms.

In a third aspect the invention is directed to a pharmaceutical composition, including: Compound A or a pharmaceutically acceptable salt thereof in combination with a AT₁ angiotensin II receptor antagonist, and a pharmaceutically acceptable carrier.

The AT₁ angiotensin II receptor antagonist may be selected from the group consisting of irbesartan, a pharmaceutically acceptable salt of irbesartan, losartan, a pharmaceutically acceptable salt of losartan, azilsartan and a pharmaceutically acceptable salt of azilsartan.

In an embodiment of the third aspect the pharmaceutical composition include Compound A or a pharmaceutically acceptable salt thereof and the AT₁ angiotensin II receptor antagonist in one single dosage form.

In another embodiment of the third aspect the pharmaceutical composition include Compound A or a pharmaceutically acceptable salt thereof and the AT₁ angiotensin II receptor antagonist in two separate dosage forms to be administered concurrently or sequentially.

In a fourth aspect the present invention provides a method for the treatment of diabetic nephropathy in a mammal in need of such treatment, including administering to the mammal suffering from diabetic nephropathy a combination of Compound A or a pharmaceutically acceptable salt thereof and an angiotensin-converting enzyme (ACE) inhibitor.

In an embodiment of the fourth aspect Compound A or a pharmaceutically acceptable salt thereof and the angiotensin-converting enzyme (ACE) inhibitor are administered in one single dosage form.

In another embodiment of the fourth aspect Compound A or a pharmaceutically acceptable salt thereof and the angiotensin-converting enzyme (ACE) inhibitor are administered concurrently or sequentially in two separate dosage forms.

In a fifth aspect the invention is directed to a pharmaceutical composition, including: Compound A or a pharmaceutically acceptable salt thereof in combination with an angiotensin-converting enzyme (ACE) inhibitor, and a pharmaceutically acceptable carrier.

The angiotensin-converting enzyme (ACE) inhibitor may be selected from the group consisting of captopril and a pharmaceutically acceptable salt of captopril.

In an embodiment of the fifth aspect the pharmaceutical composition include Compound A or a pharmaceutically acceptable salt thereof and the angiotensin-converting enzyme (ACE) inhibitor in one single dosage form.

In another embodiment of the fifth aspect the pharmaceutical composition include Compound A or a pharmaceutically acceptable salt thereof and the angiotensin-converting enzyme (ACE) inhibitor in two separate dosage forms to be administered concurrently or sequentially.

In further embodiments of the first, second and fourth aspect of the invention the mammal suffering from diabetic nephropathy is a human having any one of (a) an urinary albumin excretion rate in the range of 30-300 mg/24 h; (b) an urinary albumin excretion rate of above 300 mg/24 h; (c) an urinary albumin/creatinine ratio in the range of 30-300 mg/g; or (d) an urinary albumin/creatinine ratio of above 300 mg/g.

The details of one or more the aspects of the invention and its embodiments are set forth in the accompanying figures and description below. Other features and advantages will become apparent from the description, the figures and the claims.

### BRIEF DESCRIPTION OF THE FIGURES

FIG. 1: The effect of 8-week treatment with Compound A, roflumilast or irbesartan on urinary albumin/creatinine ratio in uninephrectomized db/db mice (8 weeks treatment starting from 4 weeks after unilateral nephrectomy)

FIG. 2: The effect of 8-week treatment with Compound A, roflumilast or irbesartan on blood level of glycosylated hemoglobin (GHb, A), plasma levels of glucose (B) and body weight (C) in uninephrectomized db/db mice (8 weeks treatment starting from 4 weeks after unilateral nephrectomy)

FIG. 3: The effect of 8-week treatment with Compound A, roflumilast or irbesartan on urinary albumin/creatinine ratio in uninephrectomized db/db mice (8 weeks treatment starting from 12 weeks after unilateral nephrectomy)

FIG. 4: The effect of 8-week treatment with Compound A or irbesartan on blood level of glycosylated hemoglobin (GHb, A), plasma levels of glucose (B) and body weight (C) in uninephrectomized db/db mice (8 weeks treatment starting from 12 weeks after unilateral nephrectomy)

FIG. 5: The effect of Compound A on intracellular cAMP level in human mesangial cells

FIG. 6: The inhibitory effect of Compound A on TGF-β-induced mRNA expression of connective tissue growth factor (CTGF) FIG. 6A and Plasminogen activator inhibitor-1 (PAI-1) FIG. 6B in human mesangial cells

FIG. 7: The inhibitory effect of Compound A on TGF-β-induced mRNA expression of type 1 collagen α1 chain (FIG. 7A) and fibronectin (FIG. 7B)

FIG. 8: Effect of 28 days treatment with Compound A in male DIO mice on Body weight and total food intake

FIG. 9: Effect of 28 days treatment with Compound A in male DIO mice on Fat Mass and Lean Mass

FIG. 10: Effect of Compound A on HbA1c levels in female db/db mice after 28 days oral treatment

### DEFINITIONS

In the present invention, the phrase “therapeutically effective amount” refers to the amount of active compound or pharmaceutical agent, or in the case of combination therapy, the combined amount of each compound or pharmaceutical agent, that elicits the biological or medicinal response that is being sought in a tissue, system, animal, individual, or human, by the researcher, veterinarian, medical doctor or other clinician, which includes one or more of the following:

Ameliorating the disease or inhibiting the disease and its progression; for example, ameliorating/inhibiting a disease, condition or disorder in an individual who is displaying the pathology or symptomatology of the disease, condition or disorder (i.e., arresting further development of the pathology and/or symptomatology or even reversing the pathology and/or symptomatology), such as in case of diabetic nephropathy, for example, mainly by decreasing one or more of (a)the urinary albumin excretion rate, (b) the urinary albumin to creatinine ratio, and/or (c) by diminishing the rate of decline in estimated glomerular filtration rate (eGFR). Ameliorating/inhibiting diabetic nephropathy might be additionally evaluated by the effect of the active compound or pharmaceutical agent on the decrease of one or more of (d) the hemoglobin A1c value, (e) the body mass index, (f) hypertension and/or the improvement of (g) lipid metabolism disorders.

As used herein, “mammal” refers to humans, mice, rats, rabbits, dogs, cats, bovines, horses, swine and monkeys, with preference given to humans.

“Early diabetic nephropathy” is defined as persistent microalbuminuria measured on at least two different occasions as an albumin excretion rate of 30 to 300 mg/24 h or equivalent thereof (for the equivalent thereof, see table below).

“Overt diabetic nephropathy” is defined as albumin excretion rate beyond the microalbuminuric range, i.e. >300 mg/24 h or equivalent thereof (for equivalent thereof, see table below).

Relationship among categories for albuminuria, proteinuria, albumin/creatinine ratio and protein/creatinine ratio:

| | Categories | | |
|---|---|---|---|
| | | Early Diabetic | Overt Diabetic |
| | Normal | Nephropathy | Nephropathy |
| | to mildly | Moderately | Severely |
| Measure | increased | increased | increased |
| Albumin excretion rate | <30 | 30-300 | >300 |
| (AER); (mg/24 h) | | | |
| Protein excretion rate | <150 | 150-500 | >500 |
| (PER); (mg/24 h) | | | |
| Albumin/creatinine | | | |
| ratio (ACR); | | | |
| (mg/mmol) | <3 | 3-30 | >30 |
| (mg/g) | <30 | 30-300 | >300 |
| Protein/creatinine | | | |
| ratio (PCR); | | | |
| (mg/mmol) | <15 | 15-50 | >50 |
| (mg/g) | <150 | 150-500 | >500 |

Albuminuria and proteinuria can be measured using excretion rates in timed urine collections, ratio of concentrations to creatinine concentration in spot urine samples. Relationships among measurement methods within a category are not exact. The relationships between AER and ACR and between PER and PCR are based on the assumption that average creatinine excretion rate is approximately 1.0 g/24 h or 10 mmol/24 h. The conversions in above table are rounded for pragmatic reasons (for an exact conversion from mg/g of creatinine to mg/mmol of creatinine, multiply by 0.113). Creatinine excretion varies with age, sex, race and diet; therefore the relationship among these categories is approximate only. ACR<10 mg/g (<1 mg/mmol) is considered normal; ACR 10-30 mg/g (1.0-3 mg/mmol) is considered high normal (Kidney International Supplements (2013), Vol 3, Issue 1; KDIGO CKD Work group).

As used herein, “pharmaceutically acceptable salt” refers to salts with bases and salts with acids. Pharmaceutically acceptable salts which may be mentioned in connection with losartan and irbesartan are the salts with alkali metals, such as sodium, potassium etc.

Pharmaceutically acceptable salts which may be mentioned in connection with Compound A are the hydrochloride, fumarate, L-tartrate edisilate, the esilate, the hydrobromide and the tosylate salt of Compound A.

“Concurrent” administration (also including “concomitant administration”), as used herein, means that both Compound A or a pharmaceutically acceptable salt thereof and irbesartan (losartan, azilsartan, captopril) or a pharmaceutically acceptable salt thereof (a) are administered to a mammal (patient) in need of the treatment in a single dosage form for simultaneous, concomitant administration or (b) are administered to a patient in need of the treatment in two separate dosage forms, and the two separate dosage forms are administered immediately one after the other. In this context, the two separate dosage forms are administered immediately one after the other, if the dosages are administered within between 0 and 15 minutes of each other; or more preferably within between 0 and 10 minutes of each other; or most preferably within between 0 and 1 minute of each other.

“Sequential” administration (also including administering “sequentially”), as used herein, means that Compound A or a pharmaceutically acceptable salt thereof is administered to the patient in need of the treatment in one dosage form and irbesartan (losartan, azilsartan, captopril) or a pharmaceutically acceptable salt thereof is administered to the mammal (patient) in need of the treatment in another separate dosage form, wherein the second dosage form is administered to the mammal (patient) in need of the treatment while the first dosage form still has an effect on the patient being treated. In a preferred embodiment of the invention the first and the second dosage form are administered in such a time interval that the effect of the combined treatment on the mammal (patient) being treated is a synergistic effect. In this context, the two separate dosage forms are administered sequentially, if the dosages are administered within between more than 15 minutes and less than 6 hours of each other; or more preferably within between more than 15 minutes and less than 4 hours; or more preferably within between more than 15 minutes and less than two hours.

### DETAILED DESCRIPTION OF THE INVENTION

The present invention provides a method for the treatment of diabetic nephropathy comprising administering to a patient in need thereof a therapeutically effective amount of either

a) a certain phosphodiesterase 4 (PDE4) inhibitor (alone) or

b) a combination of a certain phosphodiesterase 4 (PDE4) inhibitor and an AT1 angiotensin II receptor antagonist or

c) a combination of a certain phosphodiesterase 4 (PDE4) inhibitor and an angiotensin-converting enzyme inhibitor.

The phosphodiesterase 4 (PDE4) inhibitor used in the present invention is selected from the group of 5-((2R,4aR,10bR)-9-Ethoxy-2-hydroxy-8-methoxy-1,2,3,4,4a,10b-hexahydro-phenanthridin-6-yl)-1-methyl-1H-pyridin-2-one and a pharmaceutically acceptable salt thereof.

The chemical name of 5-((2R,4aR,10bR)-9-Ethoxy-2-hydroxy-8-methoxy-1,2,3,4,4a,10b-hexahydro-phenanthridin-6-yl)-1-methyl-1H-pyridin-2-one is for ease of reading at many occasions throughout this specification and the claims replaced by the expression “Compound A”.

Compound A is disclosed in U.S. Pat. No. 8,324,391, which is hereby incorporated by reference in its entirety.

Pharmaceutically acceptable salts of Compound A are disclosed in US2013/096152, which is hereby incorporated by reference in its entirety, too. Examples of pharmaceutically acceptable salts of Compound A, which may be mentioned are the hydrochloride, the fumarate, the L-tartrate, the edisilate, the esilate, the hydrobromide and the tosylate salt of Compound A. Compound A is preferably used in its free form rather than in the form of a pharmaceutically acceptable salt thereof.

Compound A may be synthesized as disclosed in U.S. Pat. No. 8,324,391.

The AT₁ angiotensin II receptor antagonist used in the present invention is preferably selected from irbesartan, a pharmaceutically acceptable salt of irbesartan, losartan, a pharmaceutically acceptable salt of losartan, azilsartan and a pharmaceuticaly acceptable salt of azilsartan.

The chemical name of irbesartan is 2-butyl-3-[p-(o-1H-tetrazol-5-ylphenyl)benzyl]-1,3-diazaspiro[4.4]non-1-en-4-one.

Irbesartan and a method for its synthesis are disclosed in U.S. Pat. No. 5,270,317, which is hereby incorporated by reference in its entirety. Various tablet formulations for irbesartan are disclosed in U.S. Pat. No. 6,342,247, which as well is hereby incorporated by reference in its entirety.

Examples of pharmaceutically acceptable salts of irbesartan, which may be mentioned are the sodium, the potassium, the hydrochloride and the hydrobromide salt of irbesartan.

The chemical name of losartan is 2-butyl-4-chloro-1-[p-(o-1H-tetrazol-5-ylphenyl)benzyl]imidazol-5-methanol.

Losartan and a method for its synthesis are disclosed in U.S. Pat. No. 5,138,069, which is hereby incorporated by reference in its entirety. A particularly preferred pharmaceutically acceptable salt of losartan is losartan potassium.

Polymorphic forms of losartan and the preparation thereof is described in U.S. Pat. No. 5,608,075, which is hereby incorporated by reference in its entirety.

Tablet formulations for losartan potassium are for example described in EP2392318, WO03/35039 and WO89/06233.

The chemical name of azilsartan is 2-ethoxy-1-{[2′-(5-oxo-4,5-dihydro-1,2,4-oxadiazol-3-yl)biphenyl-4y1]methyl}-1H-benzimidazole-7-carboxylic acid.

Azilsartan preferably is administered in form of the prodrug azilsartan medoxomil which is hydrolyzed to azilsartan in the gastrointestinal tract during absorption.

Azilsartan medoxomil and a method for its synthesis are disclosed in U.S. Pat. Nos. 7,157,584 and 7,572,920, which are hereby incorporated by reference in its entirety. The preferred pharmaceutically acceptable salt of azilsartan medoxomil is azilsartan medoxomil potassium.

Tablet formulations for azilsartan medoxomil are disclosed, for example, in U.S. Pat. No. 7,572,920. The chemical name of captopril is 1-[(25S)-3-mercapto-2-methylpropionyl]-L-proline. Captopril and a method for its synthesis are disclosed, for example, in the German patent DE2703828.

In several in vitro and in vivo (animal) experiments it has now been found that Compound A not only shows a strong ameliorating effect on parameters relevant for treatment of diabetes mellitus type 2, such as for example decrease of HbA1c and decrease of body weight, but also shows strong ameliorating effects on parameters relevant for the treatment of diabetic nephropathy, such as for example, decrease of the urinary albumin/creatinine ratio and anti-fibrotic effects in a kidney-derived cell line.

It is believed that these effects observed in the animal experiments will translate in corresponding effects in the clinical setting in humans.

In a first aspect the invention relates to a method for the treatment of diabetic nephropathy comprising administering to a mammal (patient) in need thereof a therapeutically effective amount of a phosphodiesterase 4 (PDE4) inhibitor, wherein the phosphodiesterase 4 (PDE4) inhibitor is selected from the group consisting of Compound A and a pharmaceutically acceptable salt thereof.

The AT₁ angiotensin II receptor antagonists irbesartan and losartan are both approved beside the treatment of hypertension also for the treatment of diabetic nephropathy. Thus, for example, the approved indication and usage wording in the US for irbesartan (losartan) mentions that irbesartan (losartan; in form of losartan potassium) is indicated for the treatment of diabetic nephropathy with an elevated serum creatinine and proteinura (urinary albumin to creatinine ratio 300 mg/g) in patients with type 2 diabetes and a history of hypertension.

The angiotensin-converting enzyme inhibitor captopril as well is approved beside the treatment of hypertension also for the treatment of diabetic nephropathy. The approved indication and usage wording in the US for captopril mentions that captopril is indicated for the treatment of diabetic nephropathy (proteinuria >500 mg/day) in patients with type I insulin-dependent diabetes mellitus and retinopathy.

Phosphodiesterase 4 (PDE4) inhibitors and AT1 angiotensin II receptor antagonists (angiotensin-converting enzyme inhibitors) act via different mechanisms of action; since diabetic nephropathy is a multi-factorial disease, it is believed that by taking advantage of both mechanisms of action of these different classes of compounds an even more efficacious treatment of diabetic nephropathy can be achieved.

It is furthermore believed that there is an interaction between the different mechanisms of action of these classes of compounds that results in a synergistic effect of a co-administration of Compound A (or a pharmaceutically acceptable salt thereof) with either irbesartan, losartan, azilsartan or captopril (or a pharmaceutically acceptable salt of either of these compounds) in the treatment of diabetic nephropathy.

A synergistic effect of the co-administration of Compound A (or a pharmaceutically acceptable salt thereof) with either irbesartan, losartan, azilsartan or captopril (or a pharmaceutically acceptable salt of either of these compounds) may permit to reduce the dosage of one or both of the active compounds while still obtaining clinical efficacy, thereby reducing the incidence and/or severity of adverse effects seen with the administration of one or both of these compounds.

Side effects that have been seen in connection with the administration of irbesartan/losartan potassium/azilsartan medoxomil potassium are, for example diarrhea, dyspepsia/heartburn, fatigue and dizziness.

Possible adverse effects typically seen with the administration of a phosphodiesterase 4 (PDE4) inhibitor, include diarrhea, nausea, headache, back pain, influenza, insomnia, and dizziness.

In a second aspect the invention relates to a method for the treatment of diabetic nephropathy comprising administering to a mammal (patient) in need thereof a therapeutically effective amount of a combination of a phosphodiesterase 4 (PDE4) inhibitor and an AT₁ angiotensin II receptor antagonist, wherein the phosphodiesterase 4 (PDE4) inhibitor is selected from the group consisting of Compound A and a pharmaceutically acceptable salt thereof and wherein the AT₁ angiotensin II receptor antagonist is selected from the group consisting of irbesartan, a pharmaceutically acceptable salt of irbesartan, losartan, a pharmaceutically acceptable salt of losartan, azilsartan and a pharmaceutically acceptable salt of azilsartan.

In a preferred embodiment of the first and second aspect of the invention, the phosphodiesterase 4 (PDE4) inhibitor is Compound A.

In a preferred embodiment of the second aspect of the invention the AT₁ angiotensin II receptor antagonist is selected from the group consisting of irbesartan and a pharmaceutically acceptable salt thereof.

In another preferred embodiment of the second aspect of the invention the AT₁ angiotensin II receptor antagonist is selected from the group consisting of losartan and a pharmaceutically acceptable salt of losartan.

In another preferred embodiment of the second aspect of the invention the AT₁ angiotensin II receptor antagonist is selected from the group of azilsartan and a pharmaceutically acceptable salt of azilsartan.

In another preferred embodiment of the second aspect of the invention the AT₁ angiotensin II receptor antagonist is irbesartan.

In another preferred embodiment of the second aspect of the invention the AT₁ angiotensin II receptor antagonist is losartan potassium.

In another preferred embodiment of the second aspect of the invention the AT₁ angiotensin II receptor antagonist is azilsartan.

In another preferred embodiment of the second aspect of the invention the AT1 angiotensin II receptor antagonist is azilsartan or a pharmaceutically acceptable salt thereof, and the azilsartan or the pharmaceutically acceptable salt thereof are administered to the mammal (patient) in form of azilsartan medoxomil potassium.

In another preferred embodiment of the second aspect of the invention the phosphodiesterase 4 (PDE4) inhibitor is Compound A and the AT₁ angiotensin II receptor antagonist is irbesartan.

In another preferred embodiment of the second aspect of the invention the phosphodiesterase 4 (PDE4) inhibitor is Compound A and the AT₁ angiotensin II receptor antagonist is losartan potassium.

In another preferred embodiment of the second aspect of the invention the phosphodiesterase 4 (PDE4) inhibitor is Compound A and the AT₁ angiotensin II receptor antagonist is azilsartan.

In another preferred embodiment of the second aspect of the invention the phosphodiesterase 4 (PDE4) inhibitor is Compound A and the AT₁ angiotensin II receptor antagonist is azilsartan or a pharmaceutically acceptable salt thereof, and the azilsartan or the pharmaceutically acceptable salt thereof is administered to the mammal (patient) in form of azilsartan medoxomil potassium.

The combination of the phosphodiesterase 4 (PDE4) inhibitor and the AT1 angiotensin II receptor antagonist is administered either concurrently or sequentially to the mammal (patient) in need of treatment in form of a pharmaceutical composition.

In a third aspect, the present invention is therefore directed to a pharmaceutical composition comprising
(1) a phosphodiesterase 4 (PDE4) inhibitor in combination with
(2) an AT₁ angiotensin II receptor antagonist, and
(3) at least one pharmaceutically acceptable carrier,

wherein the phosphodiesterase 4 (PDE4) inhibitor is selected from the group consisting of Compound A and a pharmaceutically acceptable salt thereof,

and wherein the AT₁ angiotensin II receptor antagonist is selected from the group consisting of irbesartan, a pharmaceutically acceptable salt of irbesartan, losartan, a pharmaceutically acceptable salt of losartan, azilsartan and a pharmaceutically acceptable salt of azilsartan.

The pharmaceutical composition can either be (a) a single dosage form containing both the phosphodiesterase 4 (PDE4) inhibitor and the AT₁ angiotensin II receptor antagonist or alternatively (b) two separate dosage forms one of which containing the phosphodiesterase 4 (PDE4) inhibitor and the other one containing the AT₁ angiotensin II receptor antagonist.

The single dosage form is used for simultaneous, concomitant administration of the two compounds of the combination, while the two separate dosage forms can be used either for simultaneous, concomitant administration, for administration one after the other or for sequential administration of the two compounds of the combination.

In a preferred embodiment of the invention the expression “pharmaceutical composition” refers in connection with combination of the two compounds to the single dosage form containing both of the two compounds.

In a preferred embodiment of the third aspect of the invention, the phosphodiesterase 4 (PDE4) inhibitor is Compound A.

In another preferred embodiment of the third aspect of the invention, the AT₁ angiotensin II receptor antagonist is selected from the group consisting of irbesartan and a pharmaceutically acceptable salt of irbesartan.

In another preferred embodiment of the third aspect of the invention, the AT₁ angiotensin II receptor antagonist is selected from the group consisting of losartan and a pharmaceutically acceptable salt of losartan.

In another preferred embodiment of the third aspect of the invention, the AT₁ angiotensin II receptor antagonist is selected from the group consisting of azilsartan and a pharmaceutically acceptable salt thereof.

In another preferred embodiment of the third aspect of the invention, the AT₁ angiotensin II receptor antagonist is irbesartan.

In another preferred embodiment of the third aspect of the invention, the AT₁ angiotensin II receptor antagonist is losartan potassium.

In another preferred embodiment of the third aspect of the invention, the AT₁ angiotensin II receptor antagonist is azilsartan.

In another preferred embodiment of the third aspect of the invention the AT1 angiotensin II receptor antagonist is azilsartan or a pharmaceutically acceptable salt thereof, and the azilsartan or the pharmaceutically acceptable salt thereof are administered to the mammal (patient) in form of azilsartan medoxomil potassium.

In another preferred embodiment of the third aspect of the invention, the phosphodiesterase 4 (PDE4) inhibitor is Compound A and the AT₁ angiotensin II receptor antagonist is irbesartan.

In another preferred embodiment of the third aspect of the invention, the phosphodiesterase 4 (PDE4) inhibitor is Compound A and the AT₁ angiotensin II receptor antagonist is losartan potassium.

In another preferred embodiment of the third aspect of the invention, the phosphodiesterase 4 (PDE4) inhibitor is Compound A and the AT₁ angiotensin II receptor antagonist is azilsartan.

In another preferred embodiment of the third aspect of the invention the phosphodiesterase 4 (PDE4) inhibitor is Compound A and the AT₁ angiotensin II receptor antagonist is azilsartan or a pharmaceutically acceptable salt thereof, and the azilsartan or the pharmaceutically acceptable salt thereof are administered to the mammal (patient) in form of azilsartan medoxomil potassium.

In a fourth aspect the invention relates to a method for the treatment of diabetic nephropathy comprising administering to a mammal (patient) in need thereof a therapeutically effective amount of a combination of a phosphodiesterase 4 (PDE4) inhibitor and an angiotensin-converting enzyme inhibitor, wherein the phosphodiesterase 4 (PDE4) inhibitor is selected from the group consisting of Compound A and a pharmaceutically acceptable salt thereof and wherein the angiotensin-converting enzyme inhibitor is selected from the group consisting of captopril and a pharmaceutically acceptable salt of captopril.

In a preferred embodiment of the fourth aspect of the invention, the phosphodiesterase 4 (PDE4) inhibitor is Compound A.

In another preferred embodiment of the fourth aspect of the invention the angiotensin-converting enzyme inhibitor is captopril.

In another preferred embodiment of the fourth aspect of the invention the phosphodiesterase 4 (PDE4) inhibitor is Compound A and the angiotensin-converting enzyme inhibitor is captopril.

The combination of the phosphodiesterase 4 (PDE4) inhibitor and the angiotensin-converting enzyme inhibitor also is administered either concurrently or sequentially to the mammal (patient) in need of treatment in form of a pharmaceutical composition.

In a fifth aspect, the present invention is therefore directed to a pharmaceutical composition comprising
(1) a phosphodiesterase 4 (PDE4) inhibitor in combination with
(2) an angiotensin-converting enzyme inhibitor, and
(3) at least one pharmaceutically acceptable carrier,

wherein the phosphodiesterase 4 (PDE4) inhibitor is selected from the group consisting of Compound A and a pharmaceutically acceptable salt thereof,

and wherein the angiotensin-converting enzyme inhibitor is selected from the group consisting of captopril and a pharmaceutically acceptable salt of captopril.

The pharmaceutical composition can either be (a) a single dosage form containing both the phosphodiesterase 4 (PDE4) inhibitor and the angiotensin-converting enzyme inhibitor or alternatively (b) two separate dosage forms one of which containing the phosphodiesterase 4 (PDE4) inhibitor and the other one containing the angiotensin-converting enzyme inhibitor.

The single dosage form is used for simultaneous, concomitant administration of the two compounds of the combination, while the two separate dosage forms can be used either for simultaneous, concomitant administration, for administration one after the other or for sequential administration of the two compounds of the combination.

In a preferred embodiment of the invention the expression “pharmaceutical composition” refers in connection with combination of the two compounds to the single dosage form containing both of the two compounds.

In a preferred embodiment of the fifth aspect of the invention, the phosphodiesterase 4 (PDE4) inhibitor is Compound A.

In another preferred embodiment of the fifth aspect of the invention, the angiotensin-converting enzyme inhibitor is selected from the group consisting of captopril and a pharmaceutically acceptable salt of captopril.

In another preferred embodiment of the fifth aspect of the invention, the angiotensin-converting enzyme inhibitor is captopril.

In another preferred embodiment of the fifth aspect of the invention, the phosphodiesterase 4 (PDE4) inhibitor is Compound A and the angiotensin-converting enzyme inhibitor is captopril.

In another preferred embodiment of the first, second and fourth aspect of the invention the mammal suffering from diabetic nephropathy is a human having an albumin excretion rate in the range of 30-300 mg/24 h.

In another preferred embodiment of the first, second and fourth aspect of the invention the mammal suffering from diabetic nephropathy is a human having an albumin excretion rate above 300 mg/24 h.

In another preferred embodiment of the first, second and fourth aspect of the invention the mammal suffering from diabetic nephropathy is human having an albumin/creatinine ratio in the range of 30-300 mg/g.

In another preferred embodiment of the first, second and fourth aspect of the invention the mammal suffering from diabetic nephropathy is a human having an albumin/creatinine ratio above 300 mg/g.

In another preferred embodiment of the first, second and fourth aspect of the invention the mammal suffering from diabetic nephropathy is a human being diagnosed to have hypertension and diabetes mellitus type 2.

In another preferred embodiment of the first, second and fourth aspect of the invention the mammal suffering from diabetic nephropathy is a human (a) having an albumin excretion rate in the range of 30-300 mg/24 h and (b) being diagnosed to have hypertension and diabetes mellitus type 2.

In another preferred embodiment of the first, second and fourth aspect of the invention the mammal suffering from diabetic nephropathy is a human (a) having an albumin excretion rate above 300 mg/24 h and (b) being diagnosed to have hypertension and diabetes mellitus type 2.

In another preferred of the first, second and fourth aspect of the invention the mammal suffering from diabetic nephropathy is a human (a) having an albumin/creatinine ratio in the range of 30-300 mg/g and (b) being diagnosed to have hypertension and diabetes mellitus type 2.

In another preferred of the first, second and fourth aspect of the invention the mammal suffering from diabetic nephropathy is a human (a) having an albumin/creatinine ratio above 300 mg/g and (b) being diagnosed to have hypertension and diabetes mellitus type 2.

Mono Therapy

In one aspect the present invention provides a method for the treatment of diabetic nephropathy comprising administering Compound A or a pharmaceutical acceptable salt thereof to the mammal (patient) in need of treatment. Compound A or a pharmaceutically acceptable salt thereof may be administered by a variety of administration routes. Administration can be, for example, oral, parenteral or transdermal. The preferred route of administration is oral.

The preferred dosage form for mono therapy is the oral dosage form. Suitable oral dosage forms include tablets, capsules, powders, pills, solutions, suspensions, emulsions, pastes and granules. The most preferred oral dosage form is a tablet.

Combination Therapy

As an alternative to mono therapy, a combination of Compound A or a pharmaceutically acceptable salt thereof and irbesartan, a pharmaceutically acceptable salt of irbesartan, losartan, a pharmaceutically acceptable salt of losartan, azilsartan, a pharmaceutically acceptable salt of azilsartan, captopril or a pharmaceutically acceptable salt of captopril may be used in the treatment of diabetic nephropathy.

For combination therapy Compound A or a pharmaceutically acceptable salt thereof may be co-administered with irbesartan (losartan, azilsartan, captopril) or a pharmaceutically acceptable salt thereof concurrently, concomitantly or sequentially. Compound A or a pharmaceutically acceptable salt thereof may be co-administered with irbesartan (losartan, azilsartan, captopril) or a pharmaceutically acceptable salt thereof by the same or different route(s) of administration. Compound A or a pharmaceutically acceptable salt thereof may be co-administered with irbesartan (losartan, azilsartan, captopril) or a pharmaceutically acceptable salt thereof in the same or different formulations, including, but not limited to:

a) a single oral dosage form containing 1) Compound A or a pharmaceutically acceptable salt thereof and 2) irbesartan (losartan, azilsartan, captopril) or a pharmaceutically acceptable salt thereof;

b) two separate oral dosage forms wherein one oral dosage form contains Compound A or a pharmaceutically acceptable salt thereof, and the other oral dosage form contains irbesartan (losartan, azilsartan, captopril) or a pharmaceutically acceptable salt thereof;

c) a single transdermal dosage form containing 1) Compound A or a pharmaceutically acceptable salt thereof and 2) irbesartan (losartan, azilsartan, captopril) or a pharmaceutically acceptable salt thereof;

d) two separate transdermal dosage forms wherein one transdermal dosage form Compound A or a pharmaceutically acceptable salt thereof, and the other transdermal dosage form contains irbesartan (losartan, azilsartan, captopril) or a pharmaceutically acceptable salt thereof;

e) a single intravenous dosage form containing 1) Compound A or a pharmaceutically acceptable salt thereof and 2) irbesartan (losartan, azilsartan, captopril) or a pharmaceutically acceptable salt thereof;

f) two separate intravenous dosage forms wherein one intravenous dosage form contains Compound A or a pharmaceutically acceptable salt thereof, and the other intravenous dosage form contains irbesartan (losartan, azilsartan, captopril) or a pharmaceutically acceptable salt thereof;

g) two separate dosage forms wherein the first dosage form contains Compound A or a pharmaceutically acceptable salt thereof, and the second dosage form contains irbesartan (losartan, azilsartan, captopril) or a pharmaceutically acceptable salt thereof, and wherein the first and the second dosage form are administered by different routes of administration.

The preferred dosage form is a single oral dosage form providing administration of 1) Compound A or a pharmaceutically acceptable salt thereof and 2) irbesartan (losartan, azilsartan, captopril) or a pharmaceutically acceptable salt thereof. Suitable oral dosage forms include tablets, capsules, powders, pills, solutions, suspensions, emulsions, pastes and granules. The most preferred oral dosage forms include tablets, each tablet containing both 1) Compound A or a pharmaceutically acceptable salt thereof and 2) irbesartan (losartan, azilsartan, captopril) or a pharmaceutically acceptable salt thereof.

If the Compound A or a pharmaceutically acceptable salt thereof and the irbesartan (losartan, azilsartan, captopril) or a pharmaceutically acceptable salt thereof are not administered in the same dosage form, each active ingredient may be administered before or after the other.

Dosage Information

Mono-Therapy

Compound A or a pharmaceutically acceptable salt thereof may be administered once daily, twice daily three times a day or four times a day. Once daily administration is particularly preferred and may take place preferably in the morning or in the evening.

Compound A may be present in an oral dosage form intended for once daily administration in any amount from 0.05 mg to 1.0 mg, such as, but not limited to 0.05, 0.1, 0.15, 0.2, 0.25, 0.3, 0.35, 0.4, 0.45, 0.5, 0.6, 0.7, 0.75, 0,8, 0.9 or 1.0 mg; more preferably in any amount from 0.05 mg to 0.5 mg, such as, but not limited to 0.05, 0.1, 0.15, 0.2, 0.25, 0.3, 0.35, 0.4, 0.45 or 0.5 mg.

If a twice daily administration is intended instead of a once daily administration the above indicated amounts of Compound A can be divided by two.

Corresponding amounts of a pharmaceutically acceptable salt of Compound A can easily be calculated by one of ordinary skill, depending on the choice of the respective salt.

Combination Therapy

The combination of Compound A or a pharmaceutically acceptable salt thereof with either irbesartan or a pharmaceutically acceptable salt thereof, losartan or a pharmaceutically acceptable salt thereof, azilsartan or a pharmaceutically acceptable salt thereof, or captopril or a pharmaceutically acceptable salt thereof may be co-administered once daily, or twice, three or four times a day.

In case of combination of Compound A or a pharmaceutically acceptable salt thereof with irbesartan (losartan, azilsartan) or a pharmaceutically acceptable salt thereof once daily co-administration is particularly preferred. Once daily co-administration may take place preferably in the morning or in the evening.

In case of combination of Compound A or a pharmaceutically acceptable salt thereof with captopril or a pharmaceutically acceptable salt thereof a three times a day co-administration is preferred.

The oral dosage forms for once daily co-administration of a combination of Compound A or a pharmaceutically acceptable salt thereof and irbesartan (losartan, azilsartan, captopril) or a pharmaceutically acceptable salt thereof, may be either in a form of
a) a single oral dosage form, with contains both Compound A or a pharmaceutically acceptable salt thereof and irbesartan (losartan, azilsartan, captopril) or a pharmaceutically acceptable salt thereof,
or in the form of
b) two separate oral dosage forms, in which one dosage form contains Compound A or a pharmaceutically acceptable salt thereof and the other dosage form contains irbesartan (losartan, azilsartan, captopril) or a pharmaceutically acceptable salt thereof).

Irbesartan may be present in any amount from about 75 mg to about 300 mg, such as for example 75 mg, 150 mg, 225 mg and 300 mg.

Losartan is preferably used in form of its potassium salt losartan potassium. Losartan potassium may be present in any amount from about 50 mg to about 100 mg.

Azilsartan is preferably used in form of the prodrug azilsartan medoxomil potassium. Azilsartan medoxomil potassium may be present in any amount from 20 to 80 mg, such as for example, 20 mg, 40 mg and 80 mg. In case, azilsartan is used as such, and not in form of azilsartan medoxomil potassium, azilsartan may be present in any amount from 10 mg to 80 mg, such as for example, 10 mg, 20 mg, 40 mg and 80 mg.

Captopril may be present in any amount from 12.5 mg to 100 mg, such as 12.5 mg, 25 mg, 50 mg and 100 mg, the preferred amount for long-term treatment of diabetic nephropathy is 25 mg three times a day.

Compound A may be present in any amount from 0.05 mg to 1.0 mg, such as, but not limited to 0.05, 0.1, 0.15, 0.2, 0.25, 0.3, 0.35, 0.4, 0.45, 0.5, 0.6, 0.7, 0.75, 0.8, 0.9 or 1.0 mg; more preferably in any amount from 0.05 mg to 0.5 mg, such as, but not limited to 0.05, 0.1, 0.15, 0.2, 0.25, 0.3, 0.35, 0.4, 0.45 or 0.5 mg.

The amount of Compound A and irbesartan, respectively, in an oral dosage form intended for once daily co-administration in an adult patient may be selected from 0.05 mg/75 mg; 0.1 mg/75 mg; 0.15 mg/75 mg; 0.2 mg/75 mg; 0.25 mg/75 mg; 0.3 mg/75 mg; 0.35 mg/75 mg; 0.4 mg/75 mg; 0.45 mg/75 mg; 0.5 mg/75 mg; 0.6 mg/75 mg; 0.7 mg/75 mg; 0.75 mg/75 mg; 0.8 mg/75 mg; 0.9 mg/75 mg; 1.0 mg/75 mg; 0.05 mg/150 mg; 0.1 mg/150 mg; 0.15 mg/150 mg; 0.2 mg/150 mg; 0.25 mg/150 mg; 0.3 mg/150 mg; 0.35 mg/150 mg; 0.4 mg/150 mg; 0.45 mg/150 mg; 0.5 mg/150 mg; 0.6 mg/150 mg; 0.7 mg/150 mg; 0.75 mg/150 mg; 0.8 mg/150 mg; 0.9 mg/150 mg; 1.0 mg/150 mg; 0.05 mg/300 mg; 0.1 mg/225 mg; 0.15 mg/225 mg; 0.2 mg/225 mg; 0.25 mg/225 mg; 0.3 mg/225 mg; 0.35 mg/225 mg; 0.4 mg/225 mg; 0.45 mg/225 mg; 0.5 mg/225 mg; 0.6 mg/225 mg; 0.7 mg/225 mg; 0.75 mg/225 mg; 0.8 mg/225 mg; 0.9 mg/225 mg; 1.0 mg/225 mg; 0.1 mg/300 mg; 0.15 mg/300 mg; 0.2 mg/300 mg; 0.25 mg/300 mg; 0.3 mg/300 mg; 0.35 mg/300 mg; 0.4 mg/300 mg; 0.45 mg/300 mg; 0.5 mg/300 mg; 0.6 mg/300 mg; 0.7 mg/300 mg; 0.75 mg/300 mg; 0.8 mg/300 mg; 0.9 mg/300 mg; and 1.0 mg/300 mg (amount Compound A/amount irbesartan).

The amount of Compound A and losartan potassium, respectively, in an oral dosage form intended for once daily co-administration in an adult patient may be selected from 0.05 mg/50 mg; 0.10 mg/50 mg;

0.15 mg/50 mg; 0.20 mg/50 mg; 0.25 mg/50 mg; 0.3 mg/50 mg; 0.35 mg/50 mg; 0.4 mg/50 mg; 0.45 mg/50 mg; 0.5 mg/50 mg; 0.6 mg/50 mg; 0.7 mg/50 mg; 0.75 mg/50 mg; 0.8 mg/50 mg; 0.9 mg/50 mg; 1.0 mg/50 mg; 0.05 mg/100 mg; 0.1 mg/100 mg; 0.15 mg/100 mg; 0.2 mg/100 mg; 0.25 mg/100 mg; 0.3 mg/100 mg; 0.35 mg/100 mg; 0.4 mg/100 mg; 0.45 mg/100 mg; 0.5 mg/100 mg; 0.6 mg/100 mg; 0.7 mg/100 mg; 0.75 mg/100 mg; 0.8 mg/100 mg; 0.9 mg/100 mg; and 1.0 mg/100 mg (amount of Compound A/amount of losartan potassium).

The amount of Compound A and azilsartan medoxomil potassium, respectively, in an oral dosage form intended for once daily co-administration in an adult patient may be selected from 0.05 mg/20 mg; 0.10 mg/20 mg; 0.15 mg/20 mg; 0.20 mg/20 mg; 0.25 mg/20 mg; 0.3 mg/20 mg; 0.35 mg/20 mg; 0.4 mg/20 mg; 0.45 mg/20 mg; 0.5 mg/20 mg; 0.6 mg/20 mg; 0.7 mg/20 mg; 0.75 mg/20 mg; 0.8 mg/20 mg; 0.9 mg/20 mg; 1.0 mg/20 mg; 0.05 mg/40 mg; 0.10 mg/40 mg; 0.15 mg/40 mg; 0.20 mg/40 mg; 0.25 mg/40 mg; 0.3 mg/40 mg; 0.35 mg/40 mg; 0.4 mg/40 mg; 0.45 mg/40 mg; 0.5 mg/40 mg; 0.6 mg/40 mg; 0.7 mg/40 mg; 0.75 mg/40 mg; 0.8 mg/40 mg; 0.9 mg/40 mg; 1.0 mg/40 mg; 0.05 mg/80 mg; 0.1 mg/80 mg; 0.15 mg/80 mg; 0.2 mg/80 mg; 0.25 mg/80 mg; 0.3 mg/80 mg; 0.35 mg/80 mg; 0.4 mg/80 mg; 0.45 mg/80 mg; 0.5 mg/80 mg; 0.6 mg/80 mg; 0.7 mg/80 mg; 0.75 mg/80 mg; 0.8 mg/80 mg; 0.9 mg/80 mg; and 1.0 mg/80 mg (amount of Compound A/amount of azilsartan medoxomil potassium).

The amount of Compound A and azilsartan, respectively, in an oral dosage form intended for once daily co-administration in an adult patient may be selected from 0.05 mg/10 mg; 0.10 mg/10 mg; 0.15 mg/10 mg; 0.20 mg/10 mg; 0.25 mg/10 mg; 0.3 mg/10 mg; 0.35 mg/10 mg; 0.4 mg/10 mg; 0.45 mg/10 mg; 0.5 mg/10 mg; 0.6 mg/10 mg; 0.7 mg/10 mg; 0.75 mg/10 mg; 0.8 mg/10 mg; 0.9 mg/10 mg; 1.0 mg/10 mg; 0.05 mg/20 mg; 0.10 mg/20 mg; 0.15 mg/20 mg; 0.20 mg/20 mg; 0.25 mg/20 mg; 0.3 mg/20 mg; 0.35 mg/20 mg; 0.4 mg/20 mg; 0.45 mg/20 mg; 0.5 mg/20 mg; 0.6 mg/20 mg; 0.7 mg/20 mg; 0.75 mg/20 mg; 0.8 mg/20 mg; 0.9 mg/20 mg; 1.0 mg/20 mg; 0.05 mg/40 mg; 0.10 mg/40 mg; 0.15 mg/40 mg; 0.20 mg/40 mg; 0.25 mg/40 mg; 0.3 mg/40 mg; 0.35 mg/40 mg; 0.4 mg/40 mg; 0.45 mg/40 mg; 0.5 mg/40 mg; 0.6 mg/40 mg; 0.7 mg/40 mg; 0.75 mg/40 mg; 0.8 mg/40 mg; 0.9 mg/40 mg; 1.0 mg/40 mg; 0.05 mg/80 mg; 0.1 mg/80 mg; 0.15 mg/80 mg; 0.2 mg/80 mg; 0.25 mg/80 mg; 0.3 mg/80 mg; 0.35 mg/80 mg; 0.4 mg/80 mg; 0.45 mg/80 mg; 0.5 mg/80 mg; 0.6 mg/80 mg; 0.7 mg/80 mg; 0.75 mg/80 mg; 0.8 mg/80 mg; 0.9 mg/80 mg; and 1.0 mg/80 mg (amount of Compound A/amount of azilsartan).

The amount of Compound A and captopril, respectively, in an oral dosage form intended for three times a day co-administration in an adult patient may be selected from 0.05 mg/25 mg; 0.10 mg/25 mg; 0.15 mg/25 mg; 0.20 mg/25 mg; 0.25 mg/25 mg; 0.3 mg/25 mg and 0.35 mg/25 mg; (amount of Compound A/amount of captopril).

If a twice daily co-administration is intended instead of a once daily co-administration the above indicated amounts of Compound A and irbesartan (losartan, azilsartan) can be divided by two.

Corresponding amounts of a pharmaceutically acceptable salt of Compound A and/or irbesartan (losartan, azilsartan, captopril) can easily be calculated by one of ordinary skill, depending on the choice of the respective salt.

Corresponding amounts of losartan or other salts than losartan potassium can easily be calculated by one of ordinary skill. Losartan potassium 50 mg contain potassium in an amount of 4.24 mg; losartan potassium 100 mg contain potassium in an amount of 8.48 mg.

In another preferred embodiment of the first aspect of the invention Compound A is administered at a daily dose of between 0.05 mg and 1 mg (more preferred between 0.05 mg and 0.5 mg) or the pharmaceutically acceptable salt of Compound A is administered at a daily dose corresponding to a Compound A daily dose of between 0.05 mg and 1.0 mg (more preferred between 0.05 mg and 0.5 mg).

In another preferred embodiment of the first aspect of the invention Compound A is administered at a daily dose selected from 0.05, 0.1, 0.15, 0.2, 0.25, 0.3, 0.35, 0.4, 0.45 and 0.5 mg or the pharmaceutically acceptable salt of Compound A is administered at a daily dose corresponding to a Compound A daily dose selected from 0.05, 0.1, 0.15, 0.2, 0.25, 0.3, 0.35, 0.4, 0.45 and 0.5 mg.

In another preferred embodiment of the second aspect of the invention Compound A is administered at a daily dose of between 0.05 mg and 1 mg or the pharmaceutically acceptable salt of Compound A is administered at a daily dose corresponding to a Compound A daily dose of between 0.05 mg and 1.0 mg and irbesartan is administered at a daily dose of between 75 mg and 300 mg or the pharmaceutically acceptable salt of irbesartan is administered at a daily dose corresponding to an irbesartan daily dose of between 75 mg and 300 mg.

In another preferred embodiment of the second aspect of the invention Compound A is administered at a daily dose selected from 0.05, 0.1, 0.15, 0.2, 0.25, 0.3, 0.35, 0.4, 0.45 and 0.5 mg or the pharmaceutically acceptable salt of Compound A is administered at a daily dose corresponding to a Compound A daily dose selected from 0.05, 0.1, 0.15, 0.2, 0.25, 0.3, 0.35, 0.4, 0.45 and 0.5 mg and irbesartan is administered at a daily dose selected from 75 mg, 150 mg, 225 mg and 300 mg or the pharmaceutically acceptable salt of irbesartan is administered at a daily dose corresponding to an irbesartan daily dose selected from 75 mg, 150 mg, 225 mg and 300 mg.

In another preferred embodiment of the second aspect of the invention Compound A is administered at a daily dose of between 0.05 mg and 1.0 mg or the pharmaceutically acceptable salt of Compound A is administered at a daily dose corresponding to a Compound A daily dose of between 0.05 mg and 1.0 mg and losartan potassium is administered at a daily dose of between 50 mg and 100 mg or losartan or another pharmaceutically acceptable salt of losartan are administered at a daily dose corresponding to a losartan potassium daily dose of between 50 mg and 100 mg.

In another preferred embodiment of the second aspect of the invention Compound A is administered at a daily dose selected from 0.05, 0.1, 0.15, 0.2, 0.25, 0.3, 0.35, 0.4, 0.45 and 0.5 mg or the pharmaceutically acceptable salt of Compound A is administered at a daily dose corresponding to a Compound A daily dose selected from 0.05, 0.1, 0.15, 0.2, 0.25, 0.3, 0.35, 0.4, 0.45 and 0.5 mg and losartan potassium is administered at a daily dose selected from 50 mg and 100 mg or losartan or another pharmaceutically acceptable salt of losartan are administered at a daily dose corresponding to a losartan potassium daily dose selected from 50 mg and 100 mg.

In another preferred embodiment of the second aspect of the invention the phosphodiesterase 4 (PDE4) inhibitor and the AT₁ angiotensin II receptor antagonist are administered in one single dosage form.

In a particularly preferred embodiment of the second aspect of the invention the phosphodiesterase 4 (PDE4) inhibitor and the AT₁ angiotensin II receptor antagonist are administered in one single oral dosage form.

In another preferred embodiment of the second aspect of the invention the phosphodiesterase 4 (PDE4) inhibitor and the AT₁ angiotensin II receptor antagonist are administered concurrently in two separate dosage forms.

In another particularly preferred embodiment of the second aspect of the invention the phosphodiesterase 4 (PDE4) inhibitor and the AT₁ angiotensin II receptor antagonist are administered concurrently in two separate oral dosage forms.

In another preferred embodiment of the second aspect of the invention the phosphodiesterase 4 (PDE4) inhibitor and the AT₁ angiotensin II receptor antagonist are administered sequentially in two separate dosage forms.

In another preferred embodiment of the second aspect of the invention the phosphodiesterase 4 (PDE4) inhibitor and the AT₁ angiotensin II receptor antagonist are administered sequentially in two separate oral dosage forms.

In a preferred embodiment of the third aspect of the invention the pharmaceutical composition comprises a) Compound A in an amount of between 0.05 mg and 1 mg or a pharmaceutically acceptable salt of Compound A in an amount corresponding to a Compound A amount of between 0.05 mg and 1.0 mg and b) irbesartan in an amount of between 75 mg and 300 mg or the pharmaceutically acceptable salt of irbesartan in an amount corresponding to an irbesartan amount of between 75 mg and 300 mg.

In another preferred embodiment of the third aspect of the invention the pharmaceutical composition comprises a) Compound A in an amount selected from 0.05, 0.1, 0.15, 0.2, 0.25, 0.3, 0.35, 0.4, 0.45 and 0.5 mg or the pharmaceutically acceptable salt of Compound A in an amount corresponding to a Compound A amount selected from 0.05, 0.1, 0.15, 0.2, 0.25, 0.3, 0.35, 0.4, 0.45 and 0.5 mg and b) irbesartan in an amount selected from 75 mg, 150 mg, 225 mg and 300 mg or a pharmaceutically acceptable salt of irbesartan in an amount corresponding to an irbesartan amount selected from 75 mg, 150 mg, 225 mg and 300 mg.

In another preferred embodiment of the third aspect of the invention the pharmaceutical composition comprises a) Compound A in an amount of between 0.05 mg and 1.0 mg or a pharmaceutically acceptable salt of Compound A in an amount corresponding to a Compound A amount of between 0.05 mg and 1.0 mg and b) losartan potassium in an amount of between 50 mg and 100 mg or losartan or another pharmaceutically acceptable salt of losartan in an amount corresponding to a losartan potassium amount of between 50 mg and 100 mg.

In another preferred embodiment of the third aspect of the invention the pharmaceutical composition comprises a) Compound A in an amount selected from 0.05, 0.1, 0.15, 0.2, 0.25, 0.3, 0.35, 0.4, 0.45 and 0.5 mg or the pharmaceutically acceptable salt of Compound A in an amount corresponding to a Compound A amount selected from 0.05, 0.1, 0.15, 0.2, 0.25, 0.3, 0.35, 0.4, 0.45 and 0.5 mg and b) losartan potassium in an amount selected from 50 mg and 100 mg or losartan or another pharmaceutically acceptable salt of losartan in an amount corresponding to a losartan potassium amount selected from 50 mg and 100 mg.

In another preferred embodiment of the third aspect of the invention the pharmaceutical composition comprises a) Compound A in an amount of between 0.05 mg and 1.0 mg or a pharmaceutically acceptable salt of Compound A in an amount corresponding to a Compound A amount of between 0.05 mg and 1.0 mg and b) azilsartan medoxomil potassium in an amount of between 20 mg and 80 mg or azilsartan medoxomil or another pharmaceutically acceptable salt of azilsartan medoxomil in an amount corresponding to a azilsartan medoxomil potassium amount of between 20 mg and 80 mg.

In another preferred embodiment of the third aspect of the invention the pharmaceutical composition comprises a) Compound A in an amount selected from 0.05, 0.1, 0.15, 0.2, 0.25, 0.3, 0.35, 0.4, 0.45 and 0.5 mg or the pharmaceutically acceptable salt of Compound A in an amount corresponding to a Compound A amount selected from 0.05, 0.1, 0.15, 0.2, 0.25, 0.3, 0.35, 0.4, 0.45 and 0.5 mg and b) azilsartan medoxomil potassium in an amount selected from 20 mg, 40 mg and 80 mg or azilsartan medoxomil or another pharmaceutically acceptable salt of azilsartan medoxomil in an amount corresponding to a azilsartan medoxomil potassium amount selected from 20 mg, 40 mg and 80 mg.

In another preferred embodiment of the third aspect of the invention the pharmaceutical composition comprises a) Compound A in an amount of between 0.05 mg and 1.0 mg or a pharmaceutically acceptable salt of Compound A in an amount corresponding to a Compound A amount of between 0.05 mg and 1.0 mg and b) azilsartan in an amount of between 10 mg and 80 mg or a pharmaceutically acceptable salt of azilsartan in an amount corresponding to an azilsartan amount of between 10 mg and 80 mg.

In another preferred embodiment of the third aspect of the invention the pharmaceutical composition comprises a) Compound A in an amount selected from 0.05, 0.1, 0.15, 0.2, 0.25, 0.3, 0.35, 0.4, 0.45 and 0.5 mg or the pharmaceutically acceptable salt of Compound A in an amount corresponding to a Compound A amount selected from 0.05, 0.1, 0.15, 0.2, 0.25, 0.3, 0.35, 0.4, 0.45 and 0.5 mg and b) azilsartan in an amount selected from 10 mg, 20 mg, 40 mg and 80 mg or a pharmaceutically acceptable salt of azilsartan in an amount corresponding to a azilsartan amount selected from 10 mg, 20 mg, 40 mg and 80 mg.

In another preferred embodiment of the third aspect of the invention the phosphodiesterase 4 (PDE4) inhibitor and the AT₁ angiotensin II receptor antagonist are in one single dosage form.

In a particularly preferred embodiment of the third aspect of the invention the phosphodiesterase 4 (PDE4) inhibitor and the AT₁ angiotensin II receptor antagonist are in one single oral dosage form.

In another preferred embodiment of the third aspect of the invention the phosphodiesterase 4 (PDE4) inhibitor and the AT₁ angiotensin II receptor antagonist are in two separate dosage forms.

In another particularly preferred embodiment of the third aspect of the invention the phosphodiesterase 4 (PDE4) inhibitor and the AT₁ angiotensin II receptor antagonist are in two separate oral dosage forms.

In another preferred embodiment of the third aspect of the invention the phosphodiesterase 4 (PDE4) inhibitor and the AT₁ angiotensin II receptor antagonist are in two separate (oral) dosage forms and the two separate (oral) dosage forms are administered concurrently.

In another preferred embodiment of the third aspect of the invention the phosphodiesterase 4 (PDE4) inhibitor and the AT₁ angiotensin II receptor antagonist are in two separate (oral) dosage forms and the two separate (oral) dosage forms are administered sequentially.

In another preferred embodiment of the fourth aspect of the invention Compound A is administered at a daily dose of between 0.05 mg and 1 mg or the pharmaceutically acceptable salt of Compound A is administered at a daily dose corresponding to a Compound A daily dose of between 0.05 mg and 1.0 mg and captopril is administered at a daily dose of between 75 mg (three times a day 25 mg) or the pharmaceutically acceptable salt of captopril administered at a daily dose corresponding to a captopril daily dose of between 75 mg (three times a day 25 mg).

In another preferred embodiment of the fourth aspect of the invention the phosphodiesterase 4 (PDE4) inhibitor and the angiotensin-converting enzyme inhibitor are administered in one single dosage form.

In a particularly preferred embodiment of the fourth aspect of the invention the phosphodiesterase 4 (PDE4) inhibitor and the angiotensin-converting enzyme inhibitor are administered in one single oral dosage form.

In another preferred embodiment of the fourth aspect of the invention the phosphodiesterase 4 (PDE4) inhibitor and the angiotensin-converting enzyme inhibitor are administered concurrently in two separate dosage forms.

In another particularly preferred embodiment of the fourth aspect of the invention the phosphodiesterase 4 (PDE4) inhibitor and the angiotensin-converting enzyme inhibitor are administered concurrently in two separate oral dosage forms.

In another preferred embodiment of the fourth aspect of the invention the phosphodiesterase 4 (PDE4) inhibitor and the angiotensin-converting enzyme inhibitor are administered sequentially in two separate dosage forms.

In another preferred embodiment of the fourth aspect of the invention the phosphodiesterase 4 (PDE4) inhibitor and the angiotensin-converting enzyme inhibitor are administered sequentially in two separate oral dosage forms.

In another preferred embodiment of the fifth aspect of the invention the phosphodiesterase 4 (PDE4) inhibitor and angiotensin-converting enzyme inhibitor are in one single dosage form.

In another preferred embodiment of the fifth aspect of the invention the phosphodiesterase 4 (PDE4) inhibitor and the angiotensin-converting enzyme inhibitor are in one single oral dosage form.

In another preferred embodiment of the fifth aspect of the invention the phosphodiesterase 4 (PDE4) inhibitor and the angiotensin-converting enzyme inhibitor are in two separate dosage forms.

In another particularly preferred embodiment of the fifth aspect of the invention the phosphodiesterase 4 (PDE4) inhibitor and the angiotensin-converting enzyme inhibitor are in two separate oral dosage forms.

In another preferred embodiment of the fifth aspect of the invention the phosphodiesterase 4 (PDE4) inhibitor and the angiotensin-converting enzyme inhibitor are in two separate (oral) dosage forms and the two separate (oral) dosage forms are administered concurrently.

In another preferred embodiment of the fifth aspect of the invention the phosphodiesterase 4 (PDE4) inhibitor and the angiotensin-converting enzyme inhibitor are in two separate (oral) dosage forms and the two separate (oral) dosage forms are administered sequentially.

Pharmaceutical Formulations and Dosage Forms

When employed as pharmaceuticals, the compounds of the invention (the PDE4 inhibitor, the AT₁ angiotensin II receptor antagonist, the angiotensin-converting enzyme inhibitor or a pharmaceutically acceptable salt thereof of either of these compounds are collectively referred to as “the compounds of the invention” in the present specification) can be administered in the form of pharmaceutical composition(s). These pharmaceutical composition(s) can be prepared in a manner well known in the pharmaceutical art and can be administered by a variety of routes. Administration can be pulmonary (e.g., by inhalation or insufflation of powders or aerosols, including by nebulizer, intratracheal, intranasal, epidermal and transdermal), oral or parenteral. Parenteral administration includes intravenous, subcutaneous, intraperitoneal or intramuscular injection, or infusion. Parenteral administration can be in the form of a single bolus dose or for example, can be by a continuous perfusion pump. Pharmaceutical composition(s) and formulations for topical administration can include: transdermal patches; conventional pharmaceutical carriers; aqueous, powder or oily bases; thickeners; and/or the like which may be necessary or desirable.

This invention also includes pharmaceutical composition(s) which contain, as the active ingredient, one or more of the compounds of the invention in combination with one or more pharmaceutically acceptable carriers. Pharmaceutically acceptable carriers known in the art can be employed. In making the pharmaceutical composition(s) of the invention, the active ingredients are typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the pharmaceutical composition(s) can be in the form of tablets, pills, powders, lozenges, sachets, cachets, elixirs, suspensions, emulsions, solutions, syrups, aerosols (as a solid or in a liquid medium), soft and hard gelatin capsules, suppositories, sterile injectable solutions, and sterile packaged powders.

The pharmaceutical composition(s) can be formulated in a unit dosage form, each dosage containing an amount of each active ingredient as described above. The term “unit dosage forms” refers to physically discrete units suitable as unitary dosages for human subjects and other mammals, each unit containing a predetermined quantity of active ingredient calculated to produce the desired therapeutic effect, in association with a suitable pharmaceutical excipient.

The compounds of the invention can be effective over a wide dosage range and are generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patients symptoms, and the like.

Pre-Clinical Studies

1) Effect of Compound A on Diabetic Nephropathy in Uninephrectomized db/db mice (8 Week Compound A Treatment Starting 4 Weeks After the Uninephrectomy)

Male 5-week-old BKS.Cg-+Lepr^{db}/+Lepr^{db} (db/db) mice were purchased from CLEA Japan, Inc. All mice were fed with a standard diet (CE-2, CLEA Japan, Inc.) and tap water ad libitum.

Compound A was obtained from Takeda GmbH, Konstanz, Germany. Irbesartan was purchased from LKT Laboratories, Inc (USA).

Experimental Protocol

In 6 week old male db/db mice an unilateral nephrectomy was performed. Under the anesthesia with isoflurane inhalation, the right kidney was removed from the mice. After 4-week recovery period, blood samples were obtained by the puncture of facial vein and blood level of glycosylated hemoglobin (GHb) was measured by automated HPLC-based GHb analyzer (HLC-723 G8, TOSOH, Japan). After the separation of plasma samples by centrifugation, also plasma levels of glucose (PG) were measured by Autoanalyzer 7180 (Hitachi, Japan). Urine samples were collected for a 8 h time-period using metabolic cages and urine creatinine level was measured by Autoanalyzer 7180. After desaltation of urine samples with PD MiniTrap G-25 column (GE healthcare, UK), urinary levels of albumin were measured by ELISA, and urinary albumin/creatinine ratio (UACR) was calculated.

Compound A (1, 3 and 10 mg/kg, QD), roflumilast (3 mg/kg, QD) and irbesartan (50 mg/kg, QD) were suspended in 0.5% methyl cellulose solution and orally administered to mice for 8 weeks starting 4 weeks after the uninephrectomy.

At the end of the 8 weeks once daily administration blood, plasma and urine samples were obtained according to the same methods as described above. GHb, PG, urinary creatinine and albumin were measured and UACR was calculated. Body weight (BW) was measured every week during the treatment.

Statistical Analysis

For evaluation of the effects of Compound A, statistical differences between vehicle- and Compound A treated groups were analyzed with two-tailed Williams' test for PG and BW and two-tailed Shirley-Williams' test for UACR and GHb. The P-values less than 0.05 were considered statistically significant in two-tailed Williams' test or Shirley-Williams' test.

For roflumilast and irbesartan, statistical differences between vehicle- and compounds-treated groups were analyzed with Student's t-test for GHb, PG and BW and Welch test for UACR. The P-values less than 0.05 were considered statistically significant in Student's t-test and Welch test.

Results

Urinary Albumin/Creatinine Ratio (UACR)

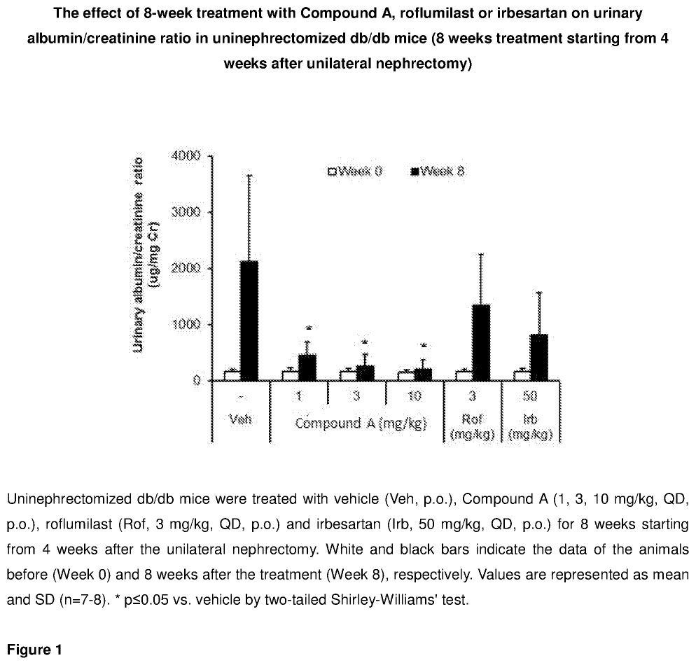

In the vehicle treated group a progressive increase of UACR was observed during the 8 weeks treatment period. Compound A significantly suppressed the increase of UACR even at the lowest tested dose (1 mg/kg) and almost completely inhibited the increase of UACR at the higher doses (3 and 10 mg/kg). Irbesartan (50 mg/kg), which was used as a positive control tended to suppress the increase of UACR, but its effect was not statistically significant in the experimental setting. (p=0.0557). The effect of roflumilast at 3 mg/kg (another PDE4 inhibitor) on UACR was also mild and was not statistically significant. In conclusion, the suppressive effect of Compound A on UACR was more potent than the effect of roflumilast on a dose basis (FIG. 1).

Glycosylated Hemoglobin (GHb) and Glucose Plasma Levels (PG)

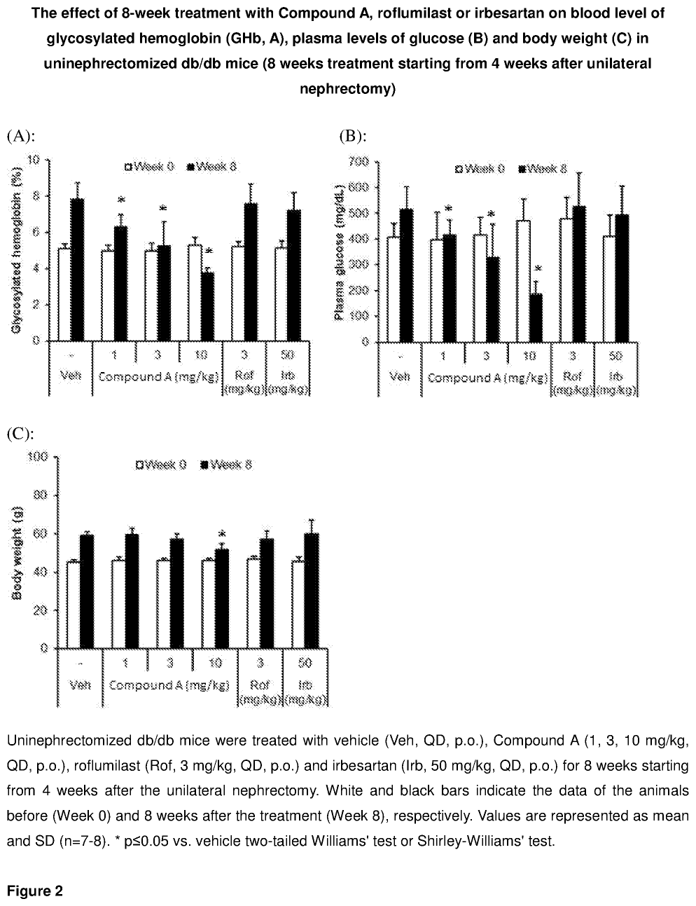

At the start the experiment, uninephrectomized db/db mice already showed a high level of GHb and PG, and both the GHb and the PG level further increased during the 8 weeks treatment period in the vehicle group. Compound A significantly inhibited the elevation of GHb and PG in a dose-dependent manner starting already from the lowest tested dose (1 mg/kg). Almost no effect compared to the vehicle group has been observed in the roflumilast (3 mg/kg) and irbesartan (50 mg/kg) treated groups with regard to the levels of GHb and PG (FIGS. 2A and 2B). In conclusion, again the effect of Compound A on GHb and PG was more potent than the effect of roflumilast on a dose basis.

Body Weight

In this obese mouse model Compound A at a dose of 10 mg/kg slightly, but statistically significantly, suppressed the increase of body weight (FIG. 2C).

2) Effect of Compound A on Diabetic Nephropathy in Uninephrectomized db/db Mice (8 Week Compound A Treatment Starting 12 Weeks After the Uninephrectomy)

Experimental Protocol

The experimental protocol is similar to the experimental protocol described above with the exception that the 8 week treatment with Compound A or irbesartan was started not already at 4 weeks after the uninephrectomy, but only 12 weeks after the uninephrectomy (i.e. when the increase of urinary albumin/creatinine ratio (UACR) is already in a much more advanced stage). Roflumilast was not used as a comparator in this experiment.

Compound A (1, 3 and 10 mg/kg, QD) and irbesartan (50 and 100 mg/kg, QD) were suspended in 0.5% methyl cellulose solution and orally administered to mice for 8 weeks starting 12 weeks after the uninephrectomy.

Statistical Analysis

For evaluation of the effects of Compound A, statistical differences between vehicle- and Compound A treated groups were analyzed with one-tailed Williams' test for GHB, PG and BW and one-tailed Shirley-Williams' test for UACR. For irbesartan, statistical differences between vehicle- and irbesartan treated groups were analyzed with one-tailed Williams' test for UACR, GHb, PG and BW. The P-values less than 0.025 were considered statistically significant in one-tailed Williams' test or one-tailed Shirley-Williams' test.

Results

Urinary Albumin/Creatinine Ratio (UACR)

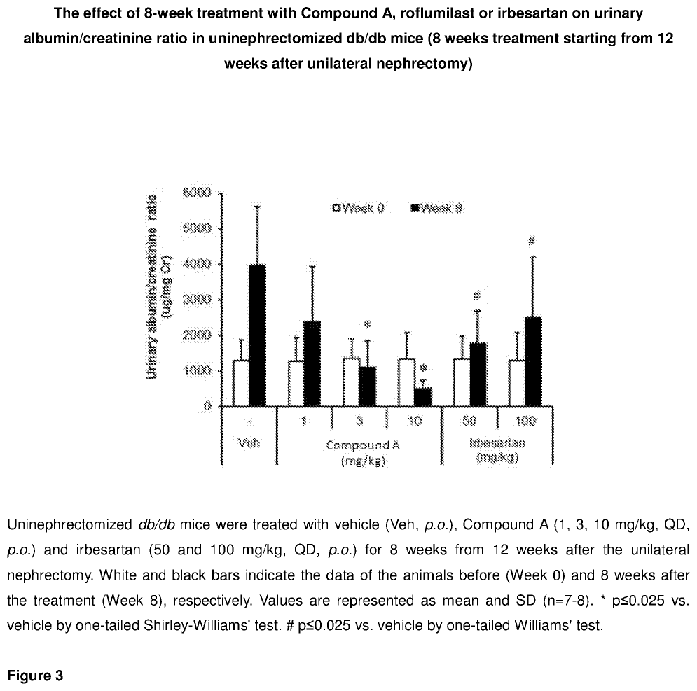

Due to the fact that in this experiment the 8 weeks treatment period started only 12 weeks after uninephrectomy, the UACR before the 8 weeks treatment period was already considerably higher than the UACR in the experiment described above, in which the 8 weeks treatment period started 4 weeks after uninephrectomy. Like in the experiment described above, in the vehicle treated group a progressive increase of UACR was observed during the 8 weeks treatment period. Compound A not only suppressed the increase of UACR in all three tested doses (1, 3 and 10 mg/kg), but also reduced the UACR in the 3 mg/kg and 10 mg/kg group below the value at the beginning of the 8 weeks treatment period. The suppression of the increase of the UACR was statistically significant in the 3 mg/kg and 10 mg/kg group. Although clear dose-dependency was not observed in irbesartan treatment group, the compound also showed significant suppression on increase in UACR at both doses tested (FIG. 3).

Glycosylated Hemoglobin (GHb) and Glucose Plasma Levels (PG)

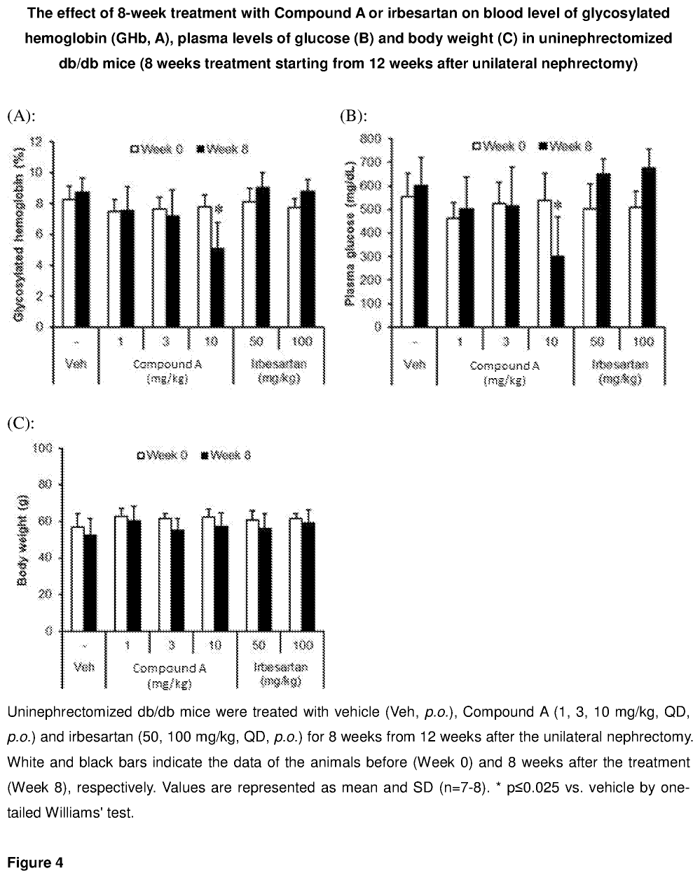

At the start of the experiment, uninephrectomized db/db mice already showed a high level for GHb and PG, and these high levels were maintained, respectively slighty increased in vehicle-treated group during 8 weeks treatment period. Compound A reduced GHb and PG in a dose-dependant manner, showing a statistical significant effect at 10 mg/kg. Irbesartan did not show inhibitory effects on these glycemic parameters (FIG. 4A and 4B).

Body Weight (BW)

Neither Compound A nor Irbesartan affected the body weight in this experimental setting (FIG. 4C).

3) The Inhibitory Effect of Compound A on TGF-β-Induced mRNA Expression of Profibrotic Factors in Human Mesangial Cells

Human mesangial cells were purchased from DS Pharma Biomedical (US). Compound A was obtained from Takeda GmbH, Konstanz, Germany. Forskolin and dimethyl sulfoxide (DMSO) were purchased from Wako Pure Chemical Industries, Ltd (Japan). Recombinant human TGF-β was purchased from R&D systems (USA).

Description of Measurements

a) Measurement of Intracellular cAMP Levels

On day 0, human mesangial cells were seeded at a cell density of 2×10⁵ cells/well in CSC complete medium (Cell Systems, USA) on type 1 collagen-coated 24-well plate (AGC TECHNO GLASS Co. Ltd., Japan) in an atmosphere of 95% air and 5% CO₂ at 37° C. On day 1, the cells were serum starved with CSC medium which is not containing serum and growth factors. On day 2, Compound A at final concentrations of 0.001, 0.01, 0.1, 1 and 10 μM or DMSO (control) was added to the culture medium. Then, thirty minutes after the addition of compound, forskolin (final concentration of 1 μM) was added. Thirty minutes after the addition of forskolin, cells were lysed in lysis buffer (Cell Signaling Technology, USA). The cell supernatants were used for the measurement of intracellular cAMP level with AlphaScreen cAMP functional assay (PerkinElmer, USA) according to the manufacture's instruction.

b) Measurement of mRNA Expression Levels

On day 0, human mesangial cells were seeded at a cell density of 2×10⁵ cells/well in CSC complete medium (Cell Systems, USA) on type 1 collagen-coated 24-well plate (AGC TECHNO GLASS Co. Ltd., Japan) in an atmosphere of 95% air and 5% CO₂ at 37° C. On day 1, the cells were serum starved with CMC medium which is not containing serum and growth factors. On day 2, Compound A at final concentrations of 0.001, 0.01, 0.1, 1 and 10 μM or DMSO (control) was added to the culture medium. Then, thirty minutes after the addition of compound, forskolin (final concentration of 1 μM) and TGF-β (final concentration of 3 ng/mL) were added. At 6 and 24 hours after the addition of forskolin and TGF-β, the medium were removed and cells were lysed in lysis buffer contained in RNeasy 96 kit (Qiagen, Germany). Total RNA was purified by RNeasy 96 kit according to the manufacture's instruction. Complimentary DNA (cDNA) was synthesized by reverse transcription reaction (High capacity cDNA reverse transcription kits, Life Technologies, USA) using isolated total RNA as a template. For the measurement of mRNA levels of connective tissue growth factor (CTGF) and plasminogen activator inhibitor-1 (PAI-1), total RNA isolated at 6 hours after forskolin/TGF-β stimulation was used. For the measurement of mRNA levels of type 1 collagen α1 chain and fibronectin, total RNA isolated at 24 hours after forskolin/TGF-β stimulation was used. mRNA expression levels were measured by quantitative real-time RT-PCR methods with TaqMan gene expression master mix and ABI PRISM 9700 (Life Technologies, USA) with target mRNA specific primer and probe sets [Human connective tissue growth factor (Hs1026927_g1); Human plasminogen activator inhibitor-1 (Hs00167155_m1); Human type 1 collagenal chain (Hs00164004_m1); Human fibronectin (HS00365052_m1); Humanglyceraldehyde 3-phosphate dehydrogenase (HS2758991_g1)].

Each mRNA expression level was calculated by ΔΔCt method according to the manufacture's instruction, and mRNA expression levels of CTGF, PAI-1, type 1 collagenal chain and fibronectin were corrected with glyceraldehyde 3-phosphate dehydrogenase (GAPDH) mRNA expression levels as an internal control.

Statistics

For evaluation of the effects of Compound A on intracellular cAMP level, statistical differences between DMSO-treatment and Compound A treatment groups were analyzed with one-tailed Williams' test. For the evaluation of the effects of Compound A on mRNA expression levels, statistical differences between forskolin/TGF-β-treatment and Compound A treatment groups were analyzed with one-tailed Williams' test. To confirm the mRNA induction by TGF-β, mRNA expression levels between untreated and TGF-β-treated group were analyzed by Students t-test. The statistical difference between TGF-β alone and TGF-β/forskolin co-treatment group was also tested by Students t-test to confirm the effect of forskolin. The P-value less than 0.025 was considered statistically significant in one-tailed Williams' test. The P-values less than 0.05 were considered statistically significant in Students t-tests.

Results

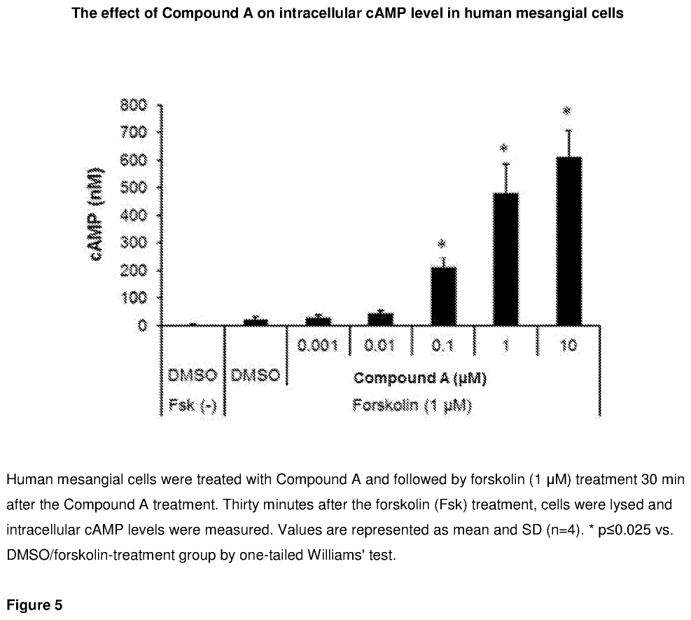

Compound A is a PDE4 selective inhibitor and has a potential to increase intracellular cAMP levels by inhibiting cAMP degrading pathway under the appropriate Gs-mediated adenylyl cyclase activation. In this in vitro assay, forskolin at 1 μM was used to activate adenylyl cyclase directly to enhance the cAMP production. In this experimental condition, forskolin alone mildly stimulated the intracellular cAMP accumulation. Co-treatment with forskolin and Compound A further enhanced intracellular cAMP accumulation in a concentration dependent manner and the significant effect was observed at 0.1 μM Compound A or above (FIG. 5). This result indicated that functional PDE4 is expressed in human mesangial cells and suggested that PDE4 plays biological roles in the cells.

To confirm the anti-fibrotic effects of Compound A in vitro, effects of Compound A on TGF-β-induced mRNA expression of profibrotic genes were investigated. Based on previous pilot studies, the maximum induction of mRNA of CTGF and PAI-1 was observed at 6 hours after the TGF-β stimulation, whereas maximum induction of mRNA of type 1 collagen and fibronectin was observed at 24 h (data not shown). In this experiment, the efficacy of Compound A on mRNA induction was investigated at 6 h after the stimulation for CTGF and PAI-1 and at 24 h after the stimulation for type 1 collagen α1 chain and fibronectin, respectively.

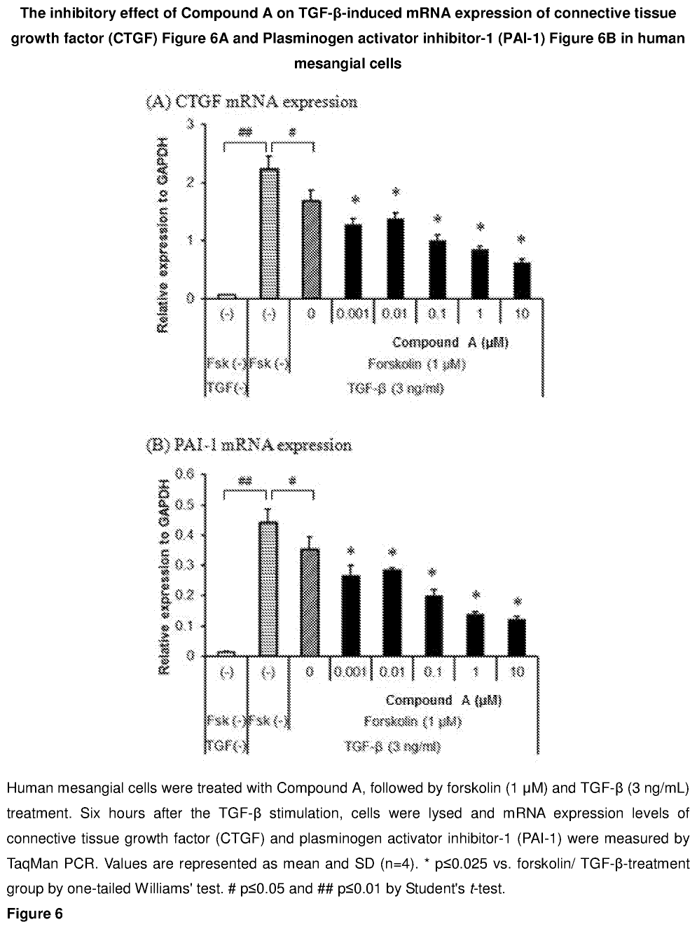

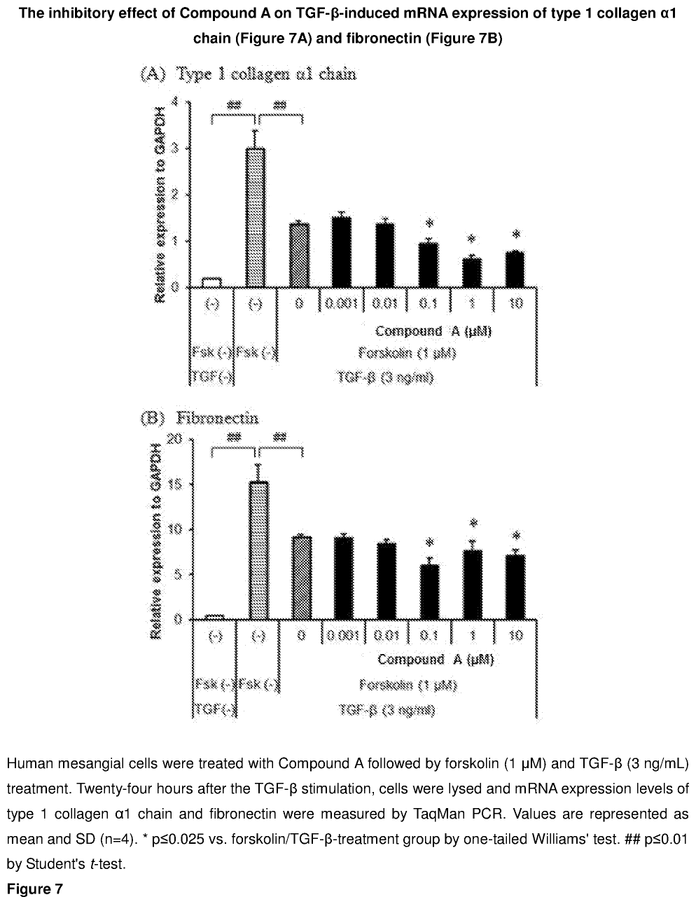

By TGF-β stimulation for 6 h, a significant mRNA induction of CTGF and PAI-1 was observed in human mesangial cells. These increases of mRNA expression were mildly, but significantly reduced by the treatment with forskolin alone, and further suppressed by the treatment with Compound A in a concentration-dependent manner (FIG. 6A and 6B). TGF-β stimulation for 24 h also significantly induced mRNA expression of both type 1 collagen α1 chain and fibronectin, and forskolin treatment alone significantly inhibited these mRNA expressions. In the presence of forskolin, Compound A further enhanced the inhibitory effect on mRNA expression of type 1 collagenal chain and fibronectin at 0.1 μM or more in human mesangial cells (FIG. 7A and 7B)

Taken together, these results demonstrated that Compound A inhibits PDE4 in human mesangial cells and enhance the accumulation of intracellular cAMP level. Furthermore, Compound A showed a robust inhibitory effect on TGF-β-induced mRNA expression of several profibrotic markers.

4) Compound A—Effect on Body Weight/Effect on Fat and Lean Mass—4 Weeks Treatment in Diet-Induced Obese (DIO) Mice

Model Description

Male C57BL/6J mice were obtained from CLEA Japan, Inc. The mice were fed High fat diet D12451 (Research diets, Inc) from 5 week to 54-week old and water ad libitum.

Experimental Protocol

All mice were housed individually in animal cages and used for the study after 2 weeks of acclimation period. Animal groups (n=7) were treated with either vehicle (0.5 w/v % methylcellulose, p.o.) or Compound A (1 mg/kg or 3 mg/kg, p.o. suspension in 0.5w/v % methylcellulose solution) in the evening once a day for 4 weeks from 50 weeks of age. Body weight was measured 2 or 3 times per week. Regarding body composition fast mass and lean mass were measured.

Measurements

Body composition (fat mass and lean mass) was measured by Echo-MRI-900 (ALOKA Japan).

Statistical Analysis

All data are presented as mean ±S.D. For evaluation of the effects of Compound A, statistical significances between vehicle (p.o.) and Compound A treated groups were analysed with one-tailed Williams' test or Shirley-Williams test when the variances among the groups were homogeneous or heterogeneous, respectively. The p-values less than 0.025 were considered statistically significant in one-tailed Williams' test or Shirley-Williams test. Body weight change from pretreatment (Day 0) was calculated using the following formula: [(BW−BW at Day 0)/BW at Day 0]×100.

Results

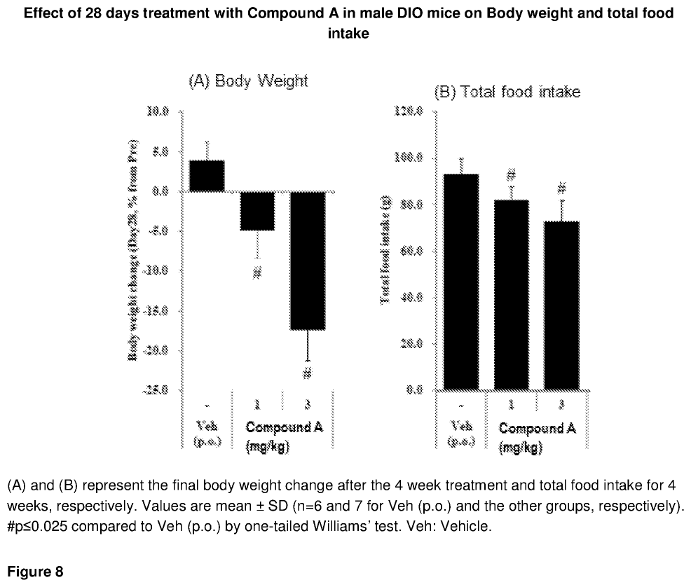

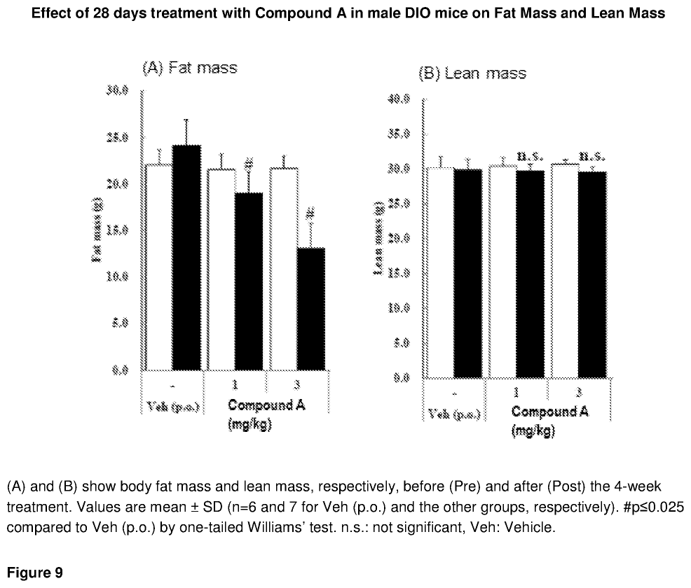

Four week-treatment with Compound A (1 and 3 mg/kg) in DIO mice dose-dependently and significantly decreased body weight (1 mg/kg; −4.9±3.5%, 3 mg/kg; −17.2±4.0%) compared to vehicle (p.o.) treated group (+3.8%±2.4%). Compound A showed a durable body weight lowering effect during the 4-week study period (FIG. 8). When corrected with vehicle (p.o.)-treated body weight change, Compound A (1 mg/kg) showed body weight reduction by −8.7±3.5%. Treatment with Compound A (1 and 3 mg/kg) dose dependently and significantly decreased fat mass (FIG. 9A) without affecting lean mass (FIG. 9B), suggesting that the body weight lowering effect of Compound A was derived from the specific reduction of fat mass.

5) Compound A—Effect on HbA1c—4 Weeks Treatment in db/db Mice Model Description

Female db/db mice were purchased from Taconic (Lille Skensved, Denmark) at 5-6 weeks of age and were maintained under standard conditions (5 animals/cage; 12 h light-dark cycle; room temperature of 22±2° C.; relative humidity of 60±15%). All mice had free access to water and standard chow (Provimi Kliba, Kaiseraugst, Switzerland). Four days upon arrival animals were randomized based on body weight and levels of glycated hemoglobin 1c (HbA1c). At 7 weeks of age, animals were treated daily by oral gavage with vehicle (4% methylcellulose) or with Compound A (composed in aqueous 4% methylcellulose) using doses of 1, 3 and 10 mg/kg s.i.d. (doses related to free base). The required dose was applied in a volume of 10 ml/kg body weight. Each dose group consisted of 10 animals. At the end of the treatment period plasma samples were isolated for determination of HbA1c levels. All experimental procedures were conducted according to the German Animal Protection Law.

HbA1c

HbA1c was analyzed from tail-tip-blood (HbA1c determination before treatment) as well as from blood collected from the retro-orbital venous plexus (HbA1c determination after 4 weeks treatment) using the Hemoglobin A1c Test (Siemens, Bad Nauheim, Germany).

Statistical Analysis

Values are presented as means ±SEM. Statistical differences were determined using one-way-ANOVA followed by a post-hoc analysis with Dunnet's correction (GraphPad Prism).

Definition of significance: n.s.=not significant (p>0.05)
*, **, ***=p<0.05, <0.01, <0.001

Results

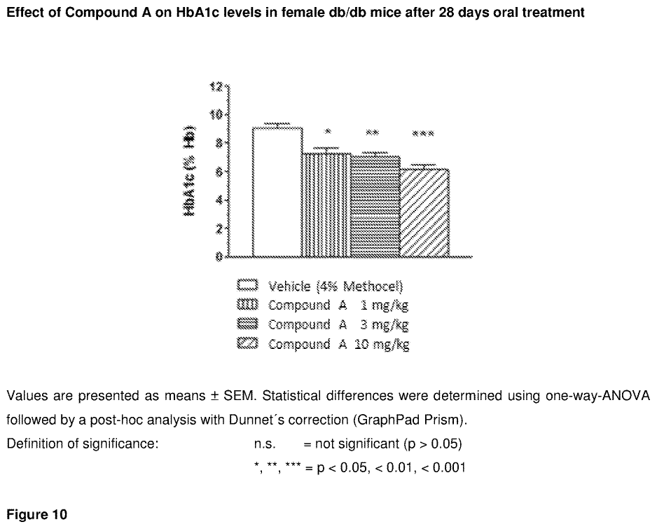

Treatment with Compound A significantly and strongly reduced HbA1c levels dose dependently at all doses tested. HbA1c levels were reduced from 9.06% (control) to 7.27 (p<0.05), 7.06 (p<0.01) and 6.16% (p<0.001) at doses of 1, 3, and 10 mg/kg of Compound A (FIG. 10).

HbA1c levels in female db/db mice after 28 days oral treatment with Compound A (n=10; doses related to free base).

| | dose (mg/kg) | mean HbA1c (%) |
|---|---|---|
| vehicle | | 9.06 |
| Compound A | 1 | 7.27* |
| | 3 | 7.06** |
| | 10 | 6.16*** |

Further aspects of the invention:
a) Use of a phosphodiesterase 4 (PDE4) inhibitor for the manufacture of a pharmaceutical composition for the treatment of diabetic nephropathy, wherein the phosphodiesterase 4 inhibitor is selected from the group consisting of Compound A and a pharmaceutically acceptable salt thereof.
b) Use according to a), wherein the phosphodiesterase 4 (PDE4) inhibitor is Compound A.
c) Use according to a), wherein Compound A is administered at a daily dose of between 0.05 and 1.0 mg or the pharmaceutically acceptable salt of Compound A is administered at a dose corresponding to a Compound A daily dose of between 0.05 mg and 1.0 mg.
d) Use according to b), wherein Compound A is administered at a daily dose of between 0.05 mg and 1.0 mg.
e) Use of a combination of
   I) a phosphodiesterase 4 (PDE4) inhibitor; and
   II) an AT₁ angiotensin II receptor antagonist,
   for the manufacture of a pharmaceutical composition for the treatment of diabetic nephropathy,
   wherein the phosphodiesterase 4 (PDE4) inhibitor is selected from the group consisting of Compound A and a pharmaceutically acceptable salt thereof,
   and wherein the AT₁ angiotensin II receptor antagonist is selected from the group consisting of irbesartan, a pharmaceutically acceptable salt of irbesartan, losartan, a pharmaceutically acceptable salt of losartan, azilsartan and a pharmaceutically acceptable salt of azilsartan.
f) Use according to e), wherein the phosphodiesterase 4 (PDE4) inhibitor is Compound A.
g) Use according to e), wherein the AT₁ angiotensin II receptor antagonist is irbesartan or a pharmaceutically acceptable salt thereof.
h) Use according to e), wherein the AT₁ angiotensin II receptor antagonist is irbesartan.
i) Use according to f), wherein the AT₁ angiotensin II receptor antagonist is irbesartan or a pharmaceutically acceptable salt thereof.
j) Use according to f), wherein the AT₁ angiotensin II receptor antagonist is irbesartan.
k) Use according to e), wherein the AT₁ angiotensin II receptor antagonist is losartan or a pharmaceutically acceptable salt thereof.
l) Use according to e), wherein the AT₁ angiotensin II receptor antagonist is losartan potassium.
m) Use according to e), wherein the AT₁ angiotensin II receptor antagonist is losartan.
n) Use according to f), wherein the AT₁ angiotensin II receptor antagonist is losartan or a pharmaceutically acceptable salt thereof.
o) Use according to f), wherein the AT₁ angiotensin II receptor antagonist is losartan potassium.
p) Use according to f), wherein the AT₁ angiotensin II receptor antagonist is losartan.
q) Use according to e), wherein the AT₁ angiotensin II receptor antagonist is azilsartan or a pharmaceutically acceptable salt thereof.
r) Use according to e), wherein the AT₁ angiotensin II receptor antagonist is azilsartan or a pharmaceutically acceptable salt thereof and azilsartan or a pharmaceutically acceptable salt thereof are administered in form of azilsartan medoxomil potassium.
s) Use according to e), wherein the AT₁ angiotensin II receptor antagonist is azilsartan.
t) Use according to f), wherein the AT₁ angiotensin II receptor antagonist is azilsartan or a pharmaceutically acceptable salt thereof.
u) Use according to f), wherein the AT₁ angiotensin II receptor antagonist is azilsartan or a pharmaceutically acceptable salt thereof and azilsartan or a pharmaceutically acceptable salt thereof are administered in form of azilsartan medoxomil potassium.
v) Use according to f), wherein the AT₁ angiotensin II receptor antagonist is azilsartan.
w) Use according to e), wherein Compound A is administered at a daily dose of between 0.05 mg and 1.0 mg or the pharmaceutically acceptable salt of Compound A is administered at a daily dose corresponding to a Compound A daily dose of between 0.05 mg and 1.0 mg.
x) Use according to f), wherein Compound A is administered at a daily dose of between 0.05 mg and 1.0 mg.
y) Use according to any one of g) to h), wherein Compound A is administered at a daily dose of between 0.05 mg and 1.0 mg or the pharmaceutically acceptable salt of Compound A is administered at a daily dose corresponding to a Compound A daily dose of between 0.05 mg and 1.0 mg, and wherein irbesartan is administered at a daily dose of between 75 mg and 300 mg or a pharmaceutically acceptable salt of irbesartan is administered at a daily dose corresponding to a irbesartan daily dose of between 75 mg and 300 mg.
z) Use according to any one of i) to j), wherein Compound A is administered at a daily dose of between 0.05 mg and 1.0 mg, and wherein irbesartan is administered at a daily dose of between 75 mg and 300 mg or a pharmaceutically acceptable salt of irbesartan is administered at a daily dose corresponding to a irbesartan daily dose of between 75 mg and 300 mg.
aa) Use according to l), wherein Compound A is administered at a daily dose of between 0.05 mg and 1.0 mg or the pharmaceutically acceptable salt of Compound A is administered at a daily dose corresponding to a Compound A daily dose of between 0.05 mg and 1.0 mg, and wherein losartan potassium is administered at a daily dose of between 50 mg and 100 mg.
bb) Use according to o), wherein Compound A is administered at a daily dose of between 0.05 mg and 1.0 mg, and wherein losartan potassium is administered at a daily dose of between 50 mg and 100 mg.
cc) Use according to any one of e) to j), y) or z), wherein the Compound A or a pharmaceutically acceptable salt thereof and the irbesartan or a pharmaceutically acceptable salt thereof are administered in one single dosage form.
dd) Use according to any one of e) to j), y) or z),wherein the Compound A or a pharmaceutically acceptable salt thereof and the irbesartan or a pharmaceutically acceptable salt thereof are administered in one single oral dosage form.
ee) Use according to any one of e) to j), y) or z), wherein the Compound A or a pharmaceutically acceptable salt thereof and the irbesartan or a pharmaceutically acceptable salt thereof are administered concurrently in two separate dosage forms.
ff) Use according to any one of e) to j), y) or z), wherein the Compound A or a pharmaceutically acceptable salt thereof and the irbesartan or a pharmaceutically acceptable salt thereof are administered concurrently in two separate oral dosage forms.
gg) Use according to any one of e) to j), y) or z), wherein the Compound A or a pharmaceutically acceptable salt thereof and the irbesartan or a pharmaceutically acceptable salt thereof are administered sequentially in two separate dosage forms.
hh) Use according to any one of e) to j), y) or z), wherein the Compound A or a pharmaceutically acceptable salt thereof and the irbesartan or a pharmaceutically acceptable salt thereof are administered sequentially in two separate oral dosage forms.
ii) Use according to any one of k) to p), aa) or bb), wherein the Compound A or a pharmaceutically acceptable salt thereof and the losartan or a pharmaceutically acceptable salt thereof are administered in one single dosage form.
jj) Use according to any one of k) to p), aa) or bb), wherein the Compound A or a pharmaceutically acceptable salt thereof and the losartan or a pharmaceutically acceptable salt thereof are administered in one single oral dosage form.
kk) Use according to any one of k) to p), aa) or bb), wherein the Compound A or a pharmaceutically acceptable salt thereof and the losartan or a pharmaceutically acceptable salt thereof are administered concurrently in two separate dosage forms.
ll) Use according to any one of k) to p), aa) or bb), wherein the Compound A or a pharmaceutically acceptable salt thereof and the losartan or a pharmaceutically acceptable salt thereof are administered concurrently in two separate oral dosage forms.
mm) Use according to any one of k) to p), aa) or bb), wherein the Compound A or a pharmaceutically acceptable salt thereof and the losartan or a pharmaceutically acceptable salt thereof are administered sequentially in two separate dosage forms.
nn) Use according to any one of k) to p), aa)or bb), wherein the Compound A or a pharmaceutically acceptable salt thereof and the losartan or a pharmaceutically acceptable salt thereof are administered sequentially in two separate oral dosage forms.
oo) Use according to any one of q) to v), wherein the Compound A or a pharmaceutically acceptable salt thereof and the azilsartan or a pharmaceutically acceptable salt thereof are administered in one single dosage form.
pp) Use according to any one of q) to v), wherein the Compound A or a pharmaceutically acceptable salt thereof and the azilsartan or a pharmaceutically acceptable salt thereof are administered in one single oral dosage form.
qq) Use according to any one of q) to v), wherein the Compound A or a pharmaceutically acceptable salt thereof and the azilsartan or a pharmaceutically acceptable salt thereof are administered concurrently in two separate dosage forms.
rr) Use according to any one of q) to v), wherein the Compound A or a pharmaceutically acceptable salt thereof and the azilsartan or a pharmaceutically acceptable salt thereof are administered concurrently in two separate oral dosage forms.
ss) Use according to any one of q) to v), wherein the Compound A or a pharmaceutically acceptable salt thereof and the azilsartan or a pharmaceutically acceptable salt thereof are administered sequentially in two separate dosage forms.
tt) Use according to any one of q) to v), wherein the Compound A or a pharmaceutically acceptable salt thereof and the azilsartan or a pharmaceutically acceptable salt thereof are administered sequentially in two separate oral dosage forms.
uu) Use according to any one of a) to d), e) to v) or w) to tt), wherein diabetic nephropathy stands for early diabetic nephropathy (urinary albumin excretion rate in the range of 30-300 mg/24 h or urinary albumin to creatinine ratio 30-300 mg/g).
vv) Use according to any one of a) to d), e) to v) or w) to tt), wherein diabetic nephropathy stands for overt diabetic nephropathy (urinary albumin excretion rate >300 mg/24 h or urinary albumin to creatinine ratio >300 mg/g).
ww) Pharmaceutical composition comprising a phosphodiesterase 4 (PDE4) inhibitor for use in the treatment of diabetic nephropathy,
   wherein the phosphodiesterase 4 (PDE4) inhibitor is selected from the group consisting of Compound A and a pharmaceutically acceptable salt thereof.
xx) Pharmaceutical composition according to ww), wherein the phosphodiesterase 4 (PDE4) inhibitor is Compound A.
yy) Pharmaceutical composition according to ww), wherein Compound A is to be administered at a daily dose of between 0.05 mg and 1.0 mg or a pharmaceutically acceptable salt of Compound A is to be administered at a dose corresponding to Compound A daily dose of between 0.05 mg and 1.0 mg.
zz) Pharmaceutical composition according to xx), wherein Compound A is to be administered at a daily dose of 0.05 mg to 1.0 mg.
aaa) A pharmaceutical composition for use in the treatment of diabetic nephropathy, which comprises:
   (1) a phosphodiesterase 4 (PDE4) inhibitor in combination with
   (2) an AT₁ angiotensin II receptor antagonist,
   wherein the phosphodiesterase 4 (PDE4) inhibitor is selected from the group consisting of Compound A and a pharmaceutically acceptable salt thereof,
   and wherein the AT₁ angiotensin II receptor antagonist is selected from the group consisting of irbesartan, a pharmaceutically acceptable salt of irbesartan, losartan, a pharmaceutically acceptable salt of losartan, azilsartan and a pharmaceutically acceptable salt of azilsartan.
bbb) The pharmaceutical composition according to aaa), wherein the phosphodiesterase 4 (PDE4) inhibitor is Compound A.
ccc) The pharmaceutical composition according to aaa), wherein the AT₁ angiotensin II receptor antagonist is irbesartan or a pharmaceutically acceptable salt thereof.
ddd) The pharmaceutical composition according to aaa), wherein the AT₁ angiotensin II receptor antagonist is irbesartan.
eee) The pharmaceutical composition according to bbb), wherein the AT₁ angiotensin II receptor antagonist is irbesartan or a pharmaceutically acceptable salt thereof.
fff) The pharmaceutical composition according to bbb), wherein the AT₁ angiotensin II receptor antagonist is irbesartan.
ggg) The pharmaceutical composition according to aaa), wherein the AT₁ angiotensin II receptor antagonist is losartan or a pharmaceutically acceptable salt thereof.
hhh) The pharmaceutical composition according to aaa), wherein the AT₁ angiotensin II receptor antagonist is losartan potassium.
iii) The pharmaceutical composition according to aaa), wherein the AT₁ angiotensin II receptor antagonist is losartan.
jjj) The pharmaceutical composition according to bbb), wherein the AT₁ angiotensin II receptor antagonist is losartan or a pharmaceutically acceptable salt thereof.
kkk) The pharmaceutical composition according to bbb), wherein the AT₁ angiotensin II receptor antagonist is losartan potassium.
lll) The pharmaceutical composition according to bbb), wherein the AT₁ angiotensin II receptor antagonist is losartan.
mmm) The pharmaceutical composition according to aaa), wherein the AT₁ angiotensin II receptor antagonist is azilsartan or a pharmaceutically acceptable salt thereof.
nnn) The pharmaceutical composition according to aaa), wherein the AT₁ angiotensin II receptor antagonist is azilsartan or a pharmaceutically acceptable salt thereof and the azilsartan or a pharmaceutically acceptable salt thereof is administered in form of azilsartan medoxomil potassium.
ooo) The pharmaceutical composition according to aaa), wherein the AT₁ angiotensin II receptor antagonist is azilsartan.
ppp) The pharmaceutical composition according to bbb), wherein the AT₁ angiotensin II receptor antagonist is azilsartan or a pharmaceutically acceptable salt thereof.
qqq) The pharmaceutical composition according to bbb), wherein the AT₁ angiotensin II receptor antagonist is azilsartan or a pharmaceutically acceptable salt thereof and the azilsartan or a pharmaceutically acceptable salt thereof is administered in form of azilsartan medoxomil potassium.
rrr) The pharmaceutical composition according to bbb), wherein the AT₁ angiotensin II receptor antagonist is azilsartan.
sss) The pharmaceutical composition according to aaa), wherein the Compound A is to be administered at a daily dose of between 0.05 mg and 1.0 mg or the pharmaceutically acceptable salt of Compound A is to be administered at a daily dose corresponding to a Compound A daily dose of between 0.05 mg and 1.0 mg.
ttt) The pharmaceutical composition according to bbb), wherein Compound A is to be administered at a daily dose of between 0.05 mg and 1.0 mg.
uuu) The pharmaceutical composition according to any one of ccc) to ddd), wherein the Compound A is to be administered at a daily dose of between 0.05 mg and 1.0 mg or the pharmaceutically acceptable salt of Compound A is to be administered at a daily dose corresponding to a Compound A daily dose of between 0.05 mg and 1.0 mg, and wherein irbesartan is to be administered at a daily dose of between 75 mg and 300 mg or a pharmaceutically acceptable salt of irbesartan is to be administered at a daily dose corresponding to a irbesartan daily dose of between 75 mg and 300 mg.
vvv) The pharmaceutical composition according to any one of eee) to fff), wherein the Compound A is to be administered at a daily dose of between 0.05 mg and 1.0 mg, and wherein irbesartan is to be administered at a daily dose of between 75 mg and 300 mg or a pharmaceutically acceptable salt of irbesartan is to be administered at a daily dose corresponding to a irbesartan daily dose of between 75 mg and 300 mg.
www) The pharmaceutical composition according to hhh), wherein the Compound A is to be administered at a daily dose of between 0.05 mg and 1.0 mg or the pharmaceutically acceptable salt of Compound A is to be administered at a daily dose corresponding to a Compound A daily dose of between 0.05 mg and 1.0 mg, and wherein losartan potassium is to be administered at a daily dose of between 50 mg and 100 mg.
xxx) The pharmaceutical composition according to kkk), wherein the Compound A is to be administered at a daily dose of between 0.05 mg and 1.0 mg, and wherein losartan potassium is to be administered at a daily dose of between 50 mg and 100 mg.
yyy) The pharmaceutical composition according to any one ccc) to fff) or uuu) to vvv), wherein the Compound A or a pharmaceutically acceptable salt thereof and the irbesartan or a pharmaceutically acceptable salt thereof are to be administered concurrently in one single dosage form.
zzz) The pharmaceutical composition according to any one ccc) to fff) or uuu) to vvv), wherein the Compound A or a pharmaceutically acceptable salt thereof and the irbesartan or a pharmaceutically acceptable salt thereof are to be administered in one single oral dosage form.
aaaa) The pharmaceutical composition according to any one ccc) to fff) or uuu) to vvv), wherein the Compound A or a pharmaceutically acceptable salt thereof and the irbesartan or a pharmaceutically acceptable salt thereof are to be administered concurrently in two separate dosage forms.
bbbb) The pharmaceutical composition according to any one ccc) to fff) or uuu) to vvv), wherein the Compound A or a pharmaceutically acceptable salt thereof and the irbesartan or a pharmaceutically acceptable salt thereof are to be administered concurrently in two separate oral dosage forms.
cccc) The pharmaceutical composition according to any one ccc) to fff) or uuu) to vvv), wherein the Compound A or a pharmaceutically acceptable salt thereof and the irbesartan or a pharmaceutically acceptable salt thereof are to be administered sequentially in two separate dosage forms.
dddd) The pharmaceutical composition according to any one ccc) to fff) or uuu) to vvv), wherein the Compound A or a pharmaceutically acceptable salt thereof and the irbesartan or a pharmaceutically acceptable salt thereof are to be administered sequentially in two separate oral dosage forms.
eeee) The pharmaceutical composition according to any one ggg) to lll) or www) to xxx), wherein the Compound A or a pharmaceutically acceptable salt thereof and the losartan or a pharmaceutically acceptable salt thereof are to be administered in one single dosage form.
ffff) The pharmaceutical composition according to any one of ggg) to lll) or www) to xxx), wherein the Compound A or a pharmaceutically acceptable salt thereof and the losartan or a pharmaceutically acceptable salt thereof are to be administered in one single oral dosage form.
gggg) The pharmaceutical composition according to any one of ggg) to lll) or www) to xxx), wherein the Compound A or a pharmaceutically acceptable salt thereof and the losartan or a pharmaceutically acceptable salt thereof are to be administered concurrently in two separate dosage forms.
hhhh) The pharmaceutical composition according to any one of ggg) to lll) or www) to xxx), wherein the Compound A or a pharmaceutically acceptable salt thereof and the losartan or a pharmaceutically acceptable salt thereof are to be administered concurrently in two separate oral dosage forms.
iiii) The pharmaceutical composition according to any one of ggg) to lll) or www) to xxx), wherein the Compound A or a pharmaceutically acceptable salt thereof and the losartan or a pharmaceutically acceptable salt thereof are to be administered sequentially in two separate dosage forms.
jjjj) The pharmaceutical composition according to any one of ggg) to lll) or www) to xxx), wherein the Compound A or a pharmaceutically acceptable salt thereof and the losartan or a pharmaceutically acceptable salt thereof are to be administered sequentially in two separate oral dosage forms.
kkkk) The pharmaceutical composition according to any one of mmm) to rrr), wherein the Compound A or a pharmaceutically acceptable salt thereof and the azilsartan or a pharmaceutically acceptable salt thereof are to be administered in one single dosage form.

llll) The pharmaceutical composition according to any one of mmm) to rrr), wherein the Compound A or a pharmaceutically acceptable salt thereof and the azilsartan or a pharmaceutically acceptable salt thereof are to be administered in one single oral dosage form.
mmmm) The pharmaceutical composition according to any one of mmm) to rrr), wherein the Compound A or a pharmaceutically acceptable salt thereof and the azilsartan or a pharmaceutically acceptable salt thereof are to be administered concurrently in two separate dosage forms.
nnnn) The pharmaceutical composition according to any one of mmm) to rrr), wherein the Compound A or a pharmaceutically acceptable salt thereof and the azilsartan or a pharmaceutically acceptable salt thereof are to be administered concurrently in two separate oral dosage forms.

oooo) The pharmaceutical composition according to any one of mmm) to rrr), wherein the Compound A or a pharmaceutically acceptable salt thereof and the azilsartan or a pharmaceutically acceptable salt thereof are to be administered sequentially in two separate dosage forms.
pppp) The pharmaceutical composition according to any one of mmm) to rrr), wherein the Compound A or a pharmaceutically acceptable salt thereof and the azilsartan or a pharmaceutically acceptable salt thereof are to be administered sequentially in two separate oral dosage forms.
qqqq) The pharmaceutical composition according to any one of ww) to zz), aaa) to xxx) or yyy) to pppp), wherein diabetic nephropathy stands for early diabetic nephropathy (urinary albumin excretion rate in the range of 30-300 mg/24 h or urinary albumin to creatinine ratio 30-300 mg/g).
rrrr) The pharmaceutical composition according to any one of ww) to zz), aaa) to xxx) or yyy) to pppp), wherein diabetic nephropathy stands for overt diabetic nephropathy (urinary albumin excretion rate >300 mg/24 h or urinary albumin to creatinine ratio >300 mg/g).
ssss) Use of a combination of
   I) a phosphodiesterase 4 (PDE4) inhibitor; and
   II) an angiotensin-converting enzyme inhibitor,
   for the manufacture of a pharmaceutical composition for the treatment of diabetic nephropathy, wherein the phosphodiesterase 4 (PDE4) inhibitor is selected from the group consisting of Compound A and a pharmaceutically acceptable salt thereof,
   and wherein the angiotensin-converting enzyme inhibitor is selected from the group consisting of captopril and a pharmaceutically acceptable salt of captopril.
tttt) The use according to ssss), wherein the phosphodiesterase 4 (PDE4) inhibitor is Compound A.
uuuu) The use according to ssss), wherein the angiotensin-converting enzyme inhibitor is captopril.
vvvv) A pharmaceutical composition for use in the treatment of diabetic nephropathy, which comprises:
   (1) a phosphodiesterase 4 (PDE4) inhibitor in combination with
   (2) an angiotensin-converting enzyme inhibitor,
   wherein the phosphodiesterase 4 (PDE4) inhibitor is selected from the group consisting of Compound A and a pharmaceutically acceptable salt thereof,
   and wherein the angiotensin-converting enzyme inhibitor is selected from the group consisting of captopril and a pharmaceutical acceptable salt of captopril.
wwww) The pharmaceutical composition according to vvvv), wherein the phosphodiesterase 4 (PDE4) inhibitor is Compound A.
xxxx) The pharmaceutical composition according to any one of vvvv) or wwww), wherein the angiotensin-converting enzyme inhibitor is captopril.

1. Pharmaceutical composition comprising a phosphodiesterase 4 (PDE4) inhibitor for use in the treatment of diabetic nephropathy,
wherein the phosphodiesterase 4 (PDE4) inhibitor is selected from the group consisting of 5-((2R,4aR,10bR)-9-Ethoxy-2-hydroxy-8-methoxy-1,2,3,4,4a,10b-hexahydro-phenanthridin-6-yl)1-methyl-1H-pyridin-2-one (hereinafter referred to as “Compound A”) and a pharmaceutically acceptable salt thereof.

2. Pharmaceutical composition according to claim 1, wherein the phosphodiesterase 4 (PDE4) inhibitor is Compound A.

3. A pharmaceutical composition for use in the treatment of diabetic nephropathy, which comprises:
(1) a phosphodiesterase 4 (PDE4) inhibitor in combination with
(2) an AT₁ angiotensin II receptor antagonist,
wherein the phosphodiesterase 4 (PDE4) inhibitor is selected from the group consisting of Compound A and a pharmaceutically acceptable salt thereof,
and wherein the AT₁ angiotensin II receptor antagonist is selected from the group consisting of irbesartan, a pharmaceutically acceptable salt of irbesartan, losartan, a pharmaceutically acceptable salt of losartan, azilsartan and a pharmaceutically acceptable salt of azilsartan.

4. The pharmaceutical composition according to claim 3, wherein the phosphodiesterase 4 (PDE4) inhibitor is Compound A.

5. The pharmaceutical composition according to claim 3, wherein the AT₁ angiotensin II receptor antagonist is irbesartan or a pharmaceutically acceptable salt thereof.

6. The pharmaceutical composition according to claim 4, wherein the AT₁ angiotensin II receptor antagonist is irbesartan or a pharmaceutically acceptable salt thereof.

7. The pharmaceutical composition according to claim 1, wherein diabetic nephropathy stands for early diabetic nephropathy (urinary albumin excretion rate in the range of 30-300 mg/24 h).

8. The pharmaceutical composition according to claim 1, wherein diabetic nephropathy stands for overt diabetic nephropathy (urinary albumin excretion rate >300 mg/24 h).

9. The pharmaceutical composition according to claim 1, wherein diabetic nephropathy stands for early diabetic nephropathy (urinary albumin to creatinine ratio 30-300 mg/g).

10. The pharmaceutical composition according to claim 1, wherein diabetic nephropathy stands for overt diabetic nephropathy (urinary albumin to creatinine ratio >300 mg/g).

11. Pharmaceutical composition comprising
(1) a phosphodiesterase 4 (PDE4) inhibitor in combination with
(2) an AT₁ angiotensin II receptor antagonist, and
(3) at least one pharmaceutically acceptable carrier,
wherein the phosphodiesterase 4 (PDE4) inhibitor is selected from the group consisting of Compound A and a pharmaceutically acceptable salt thereof,
and wherein the AT₁ angiotensin II receptor antagonist is selected from the group consisting of irbesartan, a pharmaceutically acceptable salt of irbesartan, losartan, a pharmaceutically acceptable salt of losartan, azilsartan and a pharmaceutically acceptable salt of azilsartan.

12. The pharmaceutical composition according to claim 11, wherein the phosphodiesterase 4 (PDE4) inhibitor is Compound A.

13. The pharmaceutical composition according to claim 11, wherein the AT₁ angiotensin II receptor antagonist is irbesartan or a pharmaceutically acceptable salt thereof.

14. The pharmaceutical composition according to claim 11, wherein the phosphodiesterase 4 (PDE4) inhibitor is Compound A and the AT₁ angiotensin II receptor antagonist is irbesartan or pharmaceutically acceptable salt thereof.

15. The pharmaceutical composition according to claim 11, wherein the phosphodiesterase 4 (PDE4) inhibitor and the AT₁ angiotensin II receptor antagonist are in one single oral dosage form.

16. The pharmaceutical composition according to claim 11, wherein the phosphodiesterase 4 (PDE4) inhibitor and the AT₁ angiotensin II receptor antagonist are in two separate oral dosage forms.

17. A method for the treatment of diabetic nephropathy in a mammal in need thereof, which comprises administering to a mammal suffering from diabetic nephropathy, a therapeutically effective amount of a phosphodiesterase 4 (PDE4) inhibitor,
wherein the phosphodiesterase 4 (PDE4) inhibitor is selected from the group consisting of Compound A and a pharmaceutically acceptable salt thereof.

18. The method according to claim 17, wherein the phosphodiesterase 4 (PDE4) inhibitor is Compound A.

19. A method for the treatment of diabetic nephropathy in a mammal in need thereof, which comprises: administering to a mammal suffering from diabetic nephropathy, a therapeutically effective amount of a combination of
(1) a phosphodiesterase 4 (PDE4) inhibitor; and
(2) an AT₁ angiotensin II receptor antagonist,
wherein the phosphodiesterase 4 (PDE4) inhibitor is selected from the group consisting of Compound A and a pharmaceutically acceptable salt thereof,
and wherein the AT₁ angiotensin II receptor antagonist is selected from the group consisting of irbesartan, a pharmaceutically acceptable salt of irbesartan, losartan, a pharmaceutically acceptable salt of losartan, azilsartan and a pharmaceutically acceptable salt of azilsartan.

20. The method according to claim 19, wherein the phosphodiesterase 4 (PDE4) inhibitor is Compound A.

21. The method according to claim 19, wherein the AT₁ angiotensin II receptor antagonist is irbesartan or a pharmaceutically acceptable salt thereof.

22. The method according to claim 19, wherein the phosphodiesterase 4 (PDE4) inhibitor is Compound A and the AT₁ angiotensin II receptor antagonist is irbesartan or pharmaceutically acceptable salt thereof.

23. The method according to claim 17, wherein the mammal suffering from diabetic nephropathy is a human having an urinary albumin excretion rate in the range of 30-300 mg/24 h;

24. The method according to claim 17, wherein the mammal suffering from diabetic nephropathy is a human having an urinary albumin excretion rate of above 300 mg/24 h;

25. The method according to claim 17, wherein the mammal suffering from diabetic nephropathy is a human having an urinary albumin to creatinine ratio in the range of 30-300 mg/g.

26. The method according to claim 17, wherein the mammal suffering from diabetic nephropathy is a human having an urinary albumin to creatinine ratio of above 300 mg/g.
